(19) United States
(12) Patent Application Publication
(10) Pub. No.: US 2024/0229101 A1
(43) Pub. Date: Jul. 11, 2024

(54) METHOD FOR DETECTING INTRACELLULAR ACTIVITY OF CYP ENZYMES

(71) Applicants: NATIONAL INSTITUTE OF ADVANCED INDUSTRIAL SCIENCE AND TECHNOLOGY, Tokyo (JP); OSAKA UNIVERSITY, Suita-shi, Osaka (JP)

(72) Inventors: Yasunori NAWA, Ikeda (JP); Menglu LI, Ikeda (JP); Seiichi ISHIDA, Ikeda (JP); Yasunari KANDA, Ikeda (JP); Satoshi FUJITA, Ikeda (JP); Katsumasa FUJITA, Suita (JP)

(73) Assignees: NATIONAL INSTITUTE OF ADVANCED INDUSTRIAL SCIENCE AND TECHNOLOGY, Tokyo (JP); OSAKA UNIVERSITY, Suita-shi, Osaka (JP)

(21) Appl. No.: 18/573,857

(22) PCT Filed: Jun. 22, 2022
(86) PCT No.: PCT/JP2022/024804

§ 371 Date: Dec. 22, 2023

(30) Foreign Application Priority Data
Jun. 23, 2021 (JP) 2021-104309

(51) Int. Cl. C12Q 1/26; G01N 21/65

(52) U.S. Cl. CPC C12Q 1/26; G01N 21/65; G01N 2333/90245

(57) ABSTRACT

The present invention provides a method for evaluating intracellular or extracellular enzyme activity of CYP enzyme group, including a step of measuring the number of molecules of oxidized CYP enzyme group.

### TECHNICAL FIELD

The present invention relates to a method for detecting activities of CYP enzyme group by utilizing Stokes Raman scattering signals (hereinafter to be also referred to as Raman scattering signals or Raman signals) indicating the activity of CYP metabolism enzyme group.

### BACKGROUND ART

Cytochrome p450 (CYP), known as a group of enzymes responsible for the metabolism of drugs and toxic substances, is a heme protein expressed in hepatocytes and small intestinal epithelial cells and localized in the endoplasmic reticulum (ER). In humans, 57 types of CYP genes are known. CYPs form a gene superfamily consisting of multiple molecular species (enzyme groups) with different properties including substrate specificity.

Conventionally, spectroscopy using absorption spectrum and electromagnetic paramagnetic resonance, fluorescence method using immunostaining, luminescence method using metabolic reactions of CYP, and X-ray analysis method have been used for the detection and analysis of CYP. However, in order to increase the detection sensitivity of the detection target CYP, operations for extraction, purification, or labeling of CYP protein are necessary but invasive. Since such test destroys cell tissue, it can only be applied once to one cell.

### CITATION LIST

### Non Patent Literature

### [Non Patent Literature 1]

David B. Hawkes, Gregory W. Adams, Alma L. Burlingame, Paul R. Ortiz de Montellano, James J. De Voss, “Cytochrome P450cin (CYP176A), Isolation, Expression, and Characterization”, Journal of Biological Chemistry, Volume 277, Issue 31, 2002, Pages 27725-27732, [online], [retrieved on 2021-06-04]. Retrieved from <https://doi.org/10.1074/jbc.M203382200>

### [Non Patent Literature 2]

Pelkonen, O., Turpeinen, M., Hakkola, J. et al. Inhibition and induction of human cytochrome P450 enzymes: current status. Arch Toxicol 82, 667-715 (2008), [online], [retrieved on 2021-06-04]. Retrieved from <https://doi.org/10.1007/s00204-008-0332-8>

### [Non Patent Literature 3]

Masaya Okada, Nicholas Isaac Smith, Almar Flotildes Palonpon, Hiromi Endo, Satoshi Kawata, Mikiko Sodeoka, Katsumasa Fujita, “Label-free Raman observation of cytochrome c dynamics during apoptosis”, Proceedings of the National Academy of Sciences Jan 2012, 109 (1) 28-32, [online], [retrieved on 2021-06-04]. Retrieved from <https://doi.org/10.1073/pnas.1107524108>

### [Non Patent Literature 4]

Kakita, M., Okuno, M. and Hamaguchi, H.-o., “Quantitative analysis of the redox states of cytochromes in a living L929 (NCTC) cell by resonance Raman microspectroscopy”, Journal of Biophotonics, 6, No. 3, 256-259, [online], [retrieved on 2021-06-04]. Retrieved from <https://doi.org/10.1002/jbio.201200042>

### [Non Patent Literature 5]

Athony Tu, In Raman spectroscopy in Biology; John Wiley and Sons: New York, 1982.

### [Non Patent Literature 6]

Spiro, T. G. In Biological Applications of Raman Spectroscopy; Spiro, T. G., Ed.; John Wiley and Sons: New York, 1988; Vol. III.

### [Non Patent Literature 7]

Kincaid, J. R. In The Porphyrin Handbook; Kadish, K. M., Smith, K. M., Guilard, R., Eds.; Academic Press: New York, 2000; Vol. 7, pp 225-291.

### [Non Patent Literature 8]

Zhucheng Chen, Tobias W. B. Ost, and Johannes P. M. Schelvis, “Phe393 Mutants of Cytochrome P450 BM3 with Modified Heme Redox Potentials Have Altered Heme Vinyl and Propionate Conformations”, Biochemistry, 2004, 43 (7), 1798-1808, [online], [retrieved on 2021-06-04]. Retrieved from <https://doi.org/10.1021/bi034920g>

### [Non Patent Literature 9]

Piotr J. Mak, Sang-Choul Im, Haoming Zhang, Lucy A. Waskell, and James R. Kincaid, “Resonance Raman Studies of Cytochrome P450 2B4 in Its Interactions with Substrates and Redox Partners”, Biochemistry, 2008, 47 (12), 3950-3963, [online], [retrieved on 2021-06-04]. Retrieved from <https://doi.org/10.1021/bi800034b>

### [Non Patent Literature 10]

Mak, P. J., Kaluka, D., Manyumwa, M. E., Zhang, H., Deng, T. and Kincaid, J. R., “Defining resonance Raman spectral responses to substrate binding by cytochrome P450 from Pseudomonas putida”, Biopolymers, 2008, 89: 1045-1053, [online], [retrieved on 2021-06-04]. Retrieved from <https://doi.org/10.1002/bip.21058>

### [Non Patent Literature 11]

Andrew V. Wells, Pusheng Li, Paul M. Champion, Susan A. Martinis, and Stephen G. Sligar, “Resonance Raman investigations of Escherichia coli-expressed Pseudomonas putida cytochrome P450 and P420”, Biochemistry, 1992, 31 (18), 4384-4393, [online], [retrieved on 2021-06-04]. Retrieved from <https://doi.org/10.1021/bi00133a002>

### [Non Patent Literature 12]

Piotr J. Mak, Ilia G. Denisov, Yelena V. Grinkova, Stephen G. Sligar, and James R. Kincaid, “Defining CYP3A4 Structural Responses to Substrate Binding. Raman Spectroscopic Studies of a Nanodisc-Incorporated Mammalian Cytochrome P450”, Journal of the American Chemical Society, 2011, 133 (5), 1357-1366, [online], [retrieved on 2021-06-04]. Retrieved from <https://doi.org/10.1021/ja105869p>

### [Non Patent Literature 13]

Kakita, M., Kaliaperumal, V. and Hamaguchi, H.-o. (2012), “Resonance Raman quantification of the redox state of cytochromes b and c in-vivo and in-vitro”, Journal of Biophotonics, 5, 2012, 1: 20-24, [online], [retrieved on 2021-06-04]. Retrieved from <https://doi.org/10.1002/jbio.201100087>

### [Non Patent Literature 14]

Palonpon, A., Ando, J., Yamakoshi, H. et al., “Raman and SERS microscopy for molecular imaging of live cells”, Nature Protocols, 2013, VOL.8, NO.4, 677-692, [online], [retrieved on 2021-06-10]. Retrieved from <https://doi.org/10.1038/nprot.2013.030>

### [Non Patent Literature 15]

Kouichi Yoshinari, “Drug interactions involving drug-metabolizing enzymes”, Pharmacia Biotech, 2014, vol. 50, Issue 7, p. 654-658, Publication date 2016/09/17, [online], [retrieved on 2021-06-10]. Retrieved from <https://doi.org/10.14894/faruawpsj.50.7_654>

### [Non Patent Literature 16]

Cherkas Andriy, Mondol Abdullah Saif, Ruger Jan, Urban Nadine, Popp Jurgen, Klotz Lars-Oliver, Schie Iwan W., “Label-free molecular mapping and assessment of glycogen in C. elegans”, Analyst, 2019, 144, 7, 2367-2374, [online], [retrieved on 2021-06-10]. Retrieved from <http://dx.doi.org/10.1039/C8AN02351D>

### [Non Patent Literature 17]

Neve EP, Ingelman-Sundberg M., “Cytochrome P450 proteins: retention and distribution from the endoplasmic reticulum”, Current Opinion in Drug Discovery & Development, 2010 Jan; 13(1):78-85.

### [Non Patent Literature 18]

Shu X, Lev-Ram V, Deerinck TJ, Qi Y, Ramko EB, et al. “A Genetically Encoded Tag for Correlated Light and Electron Microscopy of Intact cells, Tissues, and Organisms”, 2011, PLOS Biology 9(4): e1001041, [online], [retrieved on 2021-06-10]. Retrieved from <https://doi.org/10.1371/journal.pbio.1001041>

### [Non Patent Literature 19]

Fran Adar, Maria Erecinska, “Resonance Raman spectra of the b- And c-type cytochromes of succinate-cytochrome c reductase”, Archives of Biochemistry and Biophysics, Volume 165, Issue 2, 1974, Pages 570-580, [online], [retrieved on 2021-06-10]. Retrieved from <https://doi.org/10.1016/0003-9861(74)90284-7>

### [Non Patent Literature 20]

Naomi Kamiguchi, Eiji Aoyama, Teruaki Okuda and Toshiya Moriwaki, “A 96-Well Plate Assay for CYP4503A Induction Using Cryopreserved Human Hepatocytes”, Drug Metabolism and Disposition Nov. 1, 2010, 38 (11) 1912-1916, [online], [retrieved on 2021-06-04]. Retrieved from <https://doi.org/10.1124/dmd.109.028613>

### [Non Patent Literature 21]

J M Lehmann, D D McKee, M A Watson, T M Willson, J T Moore, S A Kliewer, “The human orphan nuclear receptor PXR is activated by compounds that regulate CYP3A4 gene expression and cause drug interactions.”, 1998-102(5)-1016-1023, [online], [retrieved on 2021-06-04]. Retrieved from <https://doi.org/10.1172/JCI3703>

### [Non Patent Literature 22]

Willson, T., Kliewer, S., “Pxr, car and drug metabolism”, Nature Reviews Drug Discovery, 1, 259-266 (2002), Apr. 1, 2002, [online], [retrieved on 2021-06-04]. Retrieved from <https://doi.org/10.1038/nrd753>

### [Non Patent Literature 23]

Aninat, Caroline, Seguin, Philippe, Descheemaeker, Pierre-Neri, Morel, Fabrice, Malledant, Yannick, Guillouzo, Andre, “Catecholamines induce an inflammatory response in human hepatocytes”, Critical Care Medicine, March 2008, Volume 36, Issue 3, p 848-854, [online], [retrieved on 2021-06-04]. Retrieved from <https://doi.org/10.1074/jbc.M203382200>

### [Non Patent Literature 24]

David M. Stresser, Marc I. Broudy, Thuy Ho, Catherine E. Cargill, Andrew P. Blanchard, Raman Sharma, Andre A. Dandeneau, Joseph J. Goodwin, Stephanie D. Turner, John C. L. Erve, Christopher J. Patten, Shangara S. Dehal and Charles L. Crespi, “Highly Selective Inhibition Of Human Cyp3a In Vitro By Azamulin And Evidence That Inhibition Is Irreversible” Drug Metabolism and Disposition, Jan. 1, 2004, 32 (1) 105-112, [online], [retrieved on 2021-06-04]. Retrieved from <https://doi.org/10.1124/dmd.32.1.105>

### SUMMARY OF INVENTION

### Technical Problem

The inventors have found the following problem. That is, in the conventional measurement of CYP expression using Raman spectroscopy, the CYP proteins were extracted and purified in order to improve the detection accuracy of the detection target CYPs. That is, invasive and destructive operations were necessary. Furthermore, with conventional techniques using such invasive and destructive operations, the enzyme activity of the CYP enzyme group within living cells has been difficult to measure.

An object of the present invention is to provide a method for detecting enzyme activities of CYP enzyme group in cells, with high resolution and without relying on invasion or labels.

### Solution to Problem

The present inventors have conducted intensive studies of the above-mentioned problem and found that
(1) the enzyme activity of CYP enzyme group is correlated with the number of molecules of oxidized CYP enzyme group
(2) the enzyme activity of CYP enzyme group within living cells can be evaluated non-invasively by measuring the number of molecules of oxidized CYP enzyme group within the cells using Raman signals, and
(3) the state of living cells (e.g., degree of drug response, degree of differentiation/undifferentiation, etc.) can be analyzed non-invasively and from multiple angles by detecting Raman signals of multiple biomolecules in the cells simultaneously. Based on these findings, the present inventors have conducted further studies and completed the present invention.

That is, the present invention provides the following.

<1> A method for evaluating enzyme activity of CYP enzyme group inside or outside a cell, comprising a step of measuring the number of molecules of oxidized CYP enzyme group.

<2> The method of <1>, wherein the method is for evaluating intracellular enzyme activity of CYP enzyme group, and wherein the step of measuring the number of molecules of the oxidized CYP enzyme group comprises
a step of irradiating an excitation light on the cells and obtaining a Raman spectrum using a photodetector, and
a step of extracting Raman scattering signals derived from the CYP enzyme group from the aforementioned Raman spectrum.

<3> The method of <1> or <2>, wherein a wavenumber of the aforementioned Raman scattering signals derived from the CYP enzyme group is in the range of wavenumber 300-600, 620-880, 920-1320, or 1320-1660 cm⁻¹.

<4> The method of <3>, wherein the aforementioned wavenumber is 1370 cm⁻¹ or 1636 cm⁻¹.

<5> The method of any of <1> to <4>, wherein the aforementioned cell is derived from any of liver, small intestine, kidney, and brain.

<6> The method of <5>, wherein the aforementioned cell is derived from the liver.

<7> The method of any of <1> to <4>, wherein the aforementioned cell is derived from a pluripotent stem cell.

<8> The method of any of <1> to <7>, further comprising a step of observing at least one selected from the group consisting of cell shape, cell size, and intracellular distribution of intracellular components in the region where the number of molecules of the CYP enzyme group is measured.

<9> The method of any of <1> to <9>, wherein the method comprises a step of further extracting Raman scattering signals derived from a substance other than the CYP enzyme group.

<10> The method of <9>, wherein the aforementioned substance other than the CYP enzyme group is at least one selected from the group consisting of reduced heme b, reduced/oxidized heme c, glycogen, reduced/oxidized cytochrome c, phenylalanine, and lipid.

<11> A method for evaluating a metabolic ability of hepatocytes, comprising the following steps:
a step of irradiating an excitation light on hepatocytes and obtaining a Raman spectrum using a photodetector, and
a step of detecting Raman signals of a biomolecule relating to metabolic ability of hepatocyte from the aforementioned Raman spectrum.

<12> The method of <11>, wherein the aforementioned biomolecule relating to metabolic ability of hepatocyte is at least one selected from the group consisting of CYP enzyme group, glycogen, cytochrome b₅, cytochrome c, lipid, and phenylalanine.

In another embodiment, the present invention is as follows.

<1′> A detection method for detecting an enzyme activity of CYP enzyme group, comprising detecting oxidized CYP enzyme group.

<2′> The method of <1′>, wherein intracellular and extracellular enzyme activities of the CYP enzyme group are detected using Raman spectrum comprising Raman scattering signals having a specific wavenumber showing an enzyme activity of the aforementioned oxidized CYP enzyme group.

<3′> The method of <1′> or <2′>, wherein the aforementioned wavenumber of the aforementioned Raman spectrum is derived from type b oxidized heme bound to the aforementioned CYP enzyme group.

<4′> The method of any of <1′> to <3′>, wherein the aforementioned specific wavenumber is in the range of wavenumber 300-600, 620-880, 920-1320, or 1320-1660 cm⁻¹.

<5′> The method of <4′>, wherein the aforementioned specific wavenumber is 1370 cm⁻¹ or 1636 cm⁻¹.

<6′> The method of any of <1′> to <5′>, wherein the aforementioned cell is a cell having CYP activity.

<7′> The method of <6′>, wherein the aforementioned cell having CYP activity is derived from any of liver, small intestine, kidney, and brain.

<8′> The method of any of <1′> to <7′>, wherein the aforementioned cell is derived from a pluripotent stem cell, for example, iPS cell or ES cell.

<9′> The method of any of <1′> to <8′>, wherein the aforementioned cell is a hepatocyte.

<10′> The method of any of <1′> to <7′>, wherein the aforementioned cell is a human liver tumor-derived cell line or a cell differentiated therefrom.

<11′> The method of any of <1′> to <10′>, wherein at least any of cell shape, cell size, and intracellular distribution of intracellular components is detected in the region where the aforementioned enzyme activity of the CYP enzyme group is detected, along with the detection of the aforementioned enzyme activity of the CYP enzyme group.

<12′> The method of any of <1′> to <11′>, wherein other molecule or the redox state thereof is detected further using other Raman scattering signals contained in the aforementioned Raman spectrum, simultaneously with the detection of the aforementioned enzyme activity of the CYP enzyme group.

<13′> The method of <12′>, wherein the aforementioned other Raman scattering signal having a specific wavenumber indicating the aforementioned other molecule is derived from any of reduced heme b, reduced/oxidized heme c, glycogen, reduced/oxidized cytochrome c, phenylalanine, and lipid.

<14′> The method of <13′>, wherein the wavenumber of the Raman scattering signal derived from the aforementioned reduced heme b is in the range of 650 to 680 cm⁻¹, 900 to 1000 cm⁻¹, 1300 to 1373 cm⁻¹, 1490 to 1500 cm−1, or 1570 to 1590 cm⁻¹.

<15′> The method of <13′> or <14′>, wherein the wavenumber of the Raman scattering signal derived from the aforementioned reduced/oxidized heme c is in the range of 590 to 640 cm⁻¹, 730 to 755 cm⁻¹, 1120 to 1130 cm⁻¹, 1310 to 1370 cm⁻¹, or 1580 to 1640 cm⁻¹.

<16′> The method of any of <13′> to <15′>, wherein the wavenumber of the Raman scattering signal derived from the aforementioned glycogen is in the range of 440 to 580 cm⁻¹, 840 to 945 cm-1, 1020 to 1660 cm⁻¹, or 2900 to 2940 cm⁻¹.

<17′> The method of any of <2′> to <16′>, wherein the method comprises a step of irradiating an excitation light on the cells and obtaining a Raman spectrum using a photodetector, and a step of extracting Raman scattering signals derived from the CYP enzyme group from the aforementioned Raman spectrum, wherein, in the aforementioned step of extracting Raman scattering signals, the peak and the wave form of a specific wavenumber of the aforementioned Raman spectrum are specified, the intersection of a line connecting both ends of the base of the aforementioned peak and a straight line drawn perpendicularly from the peak vertex to the wavenumber axis is set as the point of origin, and then the height from the aforementioned point of origin to the aforementioned peak vertex is calculated as the Raman scattering intensity.

<18′> The method of any of <2′> to <16′>, wherein the method comprises a step of irradiating an excitation light on the cells and obtaining a Raman spectrum using a photodetector, and a step of extracting Raman scattering signals derived from the CYP enzyme group from the aforementioned Raman spectrum, wherein, in the aforementioned step of extracting Raman scattering signals, the peak and the wave form of a specific wavenumber of the aforementioned Raman spectrum are specified, and the area of a region surrounded by a line connecting both ends of the base of the aforementioned peak and the waveform of the aforementioned Raman spectrum after noise removal is calculated as the Raman scattering intensity.

<19′> The method of <17′> or <18′>, further comprising evaluating at least one of the type, differentiation degree, and maturity of the cell or tissue by using the Raman scattering signals of glycogen and cytochrome c extracted from the aforementioned Raman spectrum.

<20′> A method for detecting an enzyme activity of a purified CYP protein in an environment simulating an intracellular environment, using a Raman spectrum having a specific wavenumber showing an enzyme activity of CYP enzyme group.

### [Advantageous Effects of Invention]

According to the present invention, enzyme activities of CYP enzyme group in cells can be detected with high resolution and without relying on invasion or labels.

### BRIEF DESCRIPTION OF DRAWINGS

FIG. 1 is a diagram illustrating Raman spectrum detection.

FIG. 2 is a flowchart illustrating a method for extracting Raman scattering signals.

FIG. 3 shows an example of Raman spectrum measurement results.

FIG. 4 shows Raman scattering signal extraction method 1.

FIG. 5 shows Raman scattering signal extraction method 2.

FIG. 6 shows Raman spectrum of hepatocytes in which CYP3A4 was induced.

FIG. 7 is a graph showing the measurement results of CYP3A4 by luminescence method.

FIG. 8 shows Western blotting measurement results.

FIG. 9 shows Raman spectra of hepatocytes and bile duct epithelial cells.

FIG. 10 shows Raman image constructed based on specific Raman peaks. Wavenumber 600 cm⁻¹: reduced heme c, mainly reduced cytochrome c, wavenumber 675 cm⁻¹: reduced heme b, mainly reduced cytochrome b5, wavenumber 940 cm⁻¹: glycogen, wavenumber 1636 cm⁻¹: oxidized heme b, mainly oxidized CYP enzyme group.

FIG. 11 shows Raman image constructed based on specific Raman peaks. Wavenumber 600 cm⁻¹: reduced heme c, mainly reduced cytochrome c, wavenumber 675 cm⁻¹: reduced heme b, mainly reduced cytochrome b5, wavenumber 940 cm⁻¹: glycogen, wavenumber 1000 cm^{˜1}:phenylalanine, wavenumber 1370 cm⁻¹: oxidized heme b, mainly oxidized CYP enzyme group, wavenumber 1636 cm⁻¹: oxidized heme b, mainly oxidized CYP enzyme group.

FIG. 12 shows immunostaining image of CYP3A4.

FIG. 13 shows Raman images of HepaRG cells in which CYP enzyme group was induced with Rifampicin concentrations of 0, 0.04, 0.4, and 4 piM. Wavenumber 1636 cm⁻¹: oxidized heme b, mainly oxidized CYP enzyme group.

FIG. 14 is a graph showing the activity and Raman signals of the CYP enzyme group in HepaRG cells.

FIG. 15 shows a box-whisker plot by averaging the detected Raman spectra for each cell and plotting the Raman signal at a wavenumber of 1636 cm⁻¹.

FIG. 16 is a graph showing the activity and Raman signal of the CYP enzyme group in HepaRG cells cultured using different inducing agents.

FIG. 17 shows a box-whisker plot by averaging the detected Raman spectra for each cell and plotting the Raman signals at a wavenumber of 1636 cm⁻¹.

FIG. 18 is a graph showing the activity results and Raman signals of the CYP enzyme group in HepaRG cells in which expression of the CYP enzyme group was downregulated using IL-6.

FIG. 19 shows a box-whisker plot by averaging the detected Raman spectra for each cell and plotting the Raman signals at a wavenumber of 1636 cm⁻¹.

FIG. 20 is a graph showing time course changes in the activity and Raman signal of the CYP enzyme group in HepaRG cells.

FIG. 21 shows a box-whisker plot plotting the Raman signal at a wavenumber of 1636 cm⁻¹ by averaging the detected Raman spectra for each cell.

FIG. 22 is a graph showing the activity of the CYP enzyme group in HepaRG cells in which activity of the CYP enzyme group was downregulated using Azamulin.

FIG. 23 shows a box-whisker plot by averaging the detected Raman spectra for each cell and plotting the Raman signals at a wavenumber of 1636 cm⁻¹.

FIG. 24 shows comparison photographs of Raman image and immunostaining results. Wavenumber 1636 cm⁻¹: oxidized heme b, mainly oxidized CYP enzyme group.

FIG. 25 shows Raman peaks derived from various biomolecules observed in human hepatocytes.

FIG. 26 shows Raman images of various biomolecules. Wavenumber 675 cm⁻¹:reduced heme b, mainly reduced cytochrome b5, wavenumber 940 cm⁻¹:glycogen, wavenumber 1636 cm⁻¹: oxidized heme b, mainly oxidized CYP enzyme group, wavenumber 600 cm⁻¹: reduced heme c, mainly reduced cytochrome c, wavenumber 1000 cm⁻¹:phenylalanine, wavenumber 2850 cm⁻¹:lipid.

FIG. 27 is a graph showing Raman signals of frozen primary human-derived hepatocyte (PHH) and HepaRG cells.

FIG. 28 shows images of the distribution of each molecule in PHH and HepaRG cells, obtained by visualizing the Raman signals corresponding to each spectrum. Wavenumber 600 cm⁻¹: reduced heme c, mainly reduced cytochrome c, wavenumber 675 cm⁻¹:reduced heme b, mainly reduced cytochrome b₅, wavenumber 750 cm⁻¹:all cytochromes, wavenumber 1636 cm⁻¹: oxidized heme b, mainly oxidized CYP enzyme group, wavenumber 1000 cm⁻¹:phenylalanine, mainly proteins, wavenumber 1157 cm⁻¹: carotenoids, wavenumber 1512 cm⁻¹: carotenoids.

FIG. 29 is a graph showing Raman signals of liver-like cells (HLC) and HepaRG cells.

FIG. 30 shows images of the distribution of each molecule in HLC and HepaRG cells, obtained by visualizing the Raman signals corresponding to each spectrum. Wavenumber 600 cm⁻¹: reduced heme c, mainly reduced cytochrome c, wavenumber 675 cm⁻¹:reduced heme b, mainly reduced cytochrome b5, wavenumber 750 cm⁻¹:all cytochromes, wavenumber 1636 cm⁻¹: oxidized heme b, mainly oxidized CYP enzyme group, wavenumber 1000 cm⁻¹:phenylalanine.

### DESCRIPTION OF EMBODIMENTS

The present invention is explained in detail below. The scope of the present invention is not limited to these explanations, and any matter other than the following examples can be appropriately modified and implemented without departing from the spirit of the present invention. All publications cited in this specification, such as prior art documents, publications, patent publications, and other patent documents, are incorporated herein by reference.

The present invention provides a method for evaluating enzyme activity of CYP enzyme group inside or outside a cell, including a step of measuring the number of molecules of oxidized CYP enzyme group (hereinafter sometimes referred to as “the evaluation method of the present invention”, etc.).

The present inventors have found for the first time in the world “the fact that the enzyme activity of CYP enzyme group is correlated with the number of molecules of the oxidized enzymes in the CYP enzyme group”. (Specifically, this is demonstrated by the data shown in Example 6 in the present specification). In other words, the present inventors have found that, when CYP enzyme group exists in the intracellular or extracellular environment, the enzyme activity of the CYP enzyme group can be evaluated by measuring the “number of molecules” of the oxidized CYP enzyme group by an appropriate method.

In the evaluation method of the present invention, the method used to measure the number of molecules of the oxidized CYP enzyme group in the CYP enzyme group is not particularly limited, and any method known per se may be used. Examples include, but are not limited to, infrared spectroscopy, labeling with fluorescent substance or luminescence substance, mass spectrometry, and the like. In an embodiment for evaluating the enzyme activity of CYP enzyme group in cells, Raman spectroscopy, which permits non-destructive analysis, is preferably used. One of the spectroscopic methods is, for example, identification of the redox state of cytochrome P450 using an absorption spectrum (Non Patent Literature 1).

As one embodiment of the evaluation method of the present invention, enzyme activity of CYP enzyme group in cells can be evaluated. In this embodiment, Raman spectroscopy can be utilized in the step of measuring the number of molecules of the oxidized CYP enzyme group. More specifically, the number of molecules of the oxidized CYP enzyme group can be measured by a step of irradiating an excitation light on the cells and obtaining a Raman spectrum using a photodetector, and a step of extracting Raman scattering signals derived from the CYP enzyme group from the aforementioned Raman spectrum, as a method for measuring the molecule number of the oxidized CYP enzyme group.

In this embodiment, in order to evaluate the enzyme activity of CYP enzyme group in an intracellular environment, Raman spectrum containing Raman scattering signals having specific wavenumbers is used.

CYP to be the evaluation target in the evaluation method in this embodiment is explained. CYP to be the evaluation target in one embodiment of the present invention is CYP derived from any kind of organism. Unless otherwise specified, the term “CYP enzyme group” in the present specification refers to CYP derived from any kind of organism. CYP is a heme protein containing heme as a prosthetic group.

In the evaluation method according to one embodiment of the present invention, the “redox state” derived from the molecular structure specific to CYP molecule is detected. The molecular structure specific to CYP molecule is as follows. There are subtypes (type a, type b, etc.) depending on the porphyrin structure of the heme, and CYP has a type b heme structure. CYPs form a gene superfamily consisting of multiple molecular species (enzyme groups) different in properties including substrate specificity. In human, 57 types of CYP genes are known. Among the 57 types of CYP genes, the main CYP enzyme group involved in the metabolism of drugs and toxic substances include CYP3A4, CYP1A2, CYP2B6, and the like. In human, CYP enzymes are expressed mainly in hepatocytes of the liver and epithelial cells of the small intestine, and are localized in the endoplasmic reticulum (ER). In particular, it is known that expression of CYP3A4 is highest in the human liver, accounting for 35% of the entire CYP family (Non Patent Literature 2). In the evaluation method according to one embodiment of the present invention, the total intensity of is Raman scattered light at a specific Raman shift is detected for all CYP molecules included in the CYP enzyme group, without distinguishing between the types of molecules.

In one embodiment, the “CYP enzyme group” whose activity is evaluated by the evaluation method of the present invention refers to 57 types of human CYP enzymes. In another embodiment, the “CYP enzyme group” may be CYP enzymes whose expression is induced by an inducing agent known to induce expression of the CYP enzyme group, among the 57 types of human CYP enzymes. (Specific examples of such inducing agent include, but are not limited to, Rifampicin, Phenytoin, Carbamazepine, and Dexamethasone. The present inventors confirmed that the four inducing agents recited as examples induce gene expression of at least one of four CYP enzymes selected from the group consisting of CYP3A4, CYP1A2, CYP2B6, and CYP2C9. CYP enzyme whose expression is induced by an inducing agent may be at least one selected from the group consisting of CYP3A4, CYP1A2, CYP2B6, and CYP2C9.

In one embodiment of the present invention, the “CYP enzyme group” may be any of the following (1) to (15):
(1) human CYP3A4
(2) human CYP1A2
(3) human CYP2B6
(4) human CYP2C9
(5) human CYP3A4 and human CYP1A2
(6) human CYP3A4 and human CYP2B6
(7) human CYP3A4 and human CYP2C9
(8) human CYP1A2 and human CYP2B6
(9) human CYP1A2 and human CYP2C9
(10) human CYP2B6 and human CYP2C9
(11) human CYP3A4 and human CYP1A2 and human CYP2B6
(12) human CYP3A4 and human CYP1A2 and human CYP2C9
(13) human CYP3A4 and human CYP2B6 and human CYP2C9
(14) human CYP1A2 and human CYP2B6 and human CYP2C9
(15) human CYP3A4 and human CYP1A2 and human CYP2B6 human CYP2C9.

The evaluation method of the present invention can evaluate enzyme activity of metabolism by not only human CYP3A4 but also CYPs derived from any kind of organisms. Heme b is an iron porphyrin complex with an iron atom as the central metal.

One highly reactive oxygen atom is bonded to the iron atom of heme b. CYP introduces one oxygen atom into a target compound, such as a drug, toxic substance, or the like, and oxidizes the compound, thereby increasing the water solubility of the compound and promoting excretion thereof from the body.

Therefore, the iron atom of heme b reversibly changes between the trivalent oxidized form (Fe³⁺) and the divalent reduced form (Fe²⁺) by the oxidation reduction reaction. In the evaluation method of the present invention, the metabolic enzyme activity of the CYP enzyme group is evaluated based on the measurement results of the redox state of the CYP enzyme group.

The detection principle used in the evaluation method in this embodiment is Raman spectroscopy. Raman spectroscopy permits the measurement of CYP enzyme activity in a non-destructive, non-staining, and non-invasive manner. In Raman spectroscopy, when an excitation light is irradiated on the object to be measured, Raman scattered light is generated. The spectrum obtained from Raman scattered light has peaks at plural specific wavenumbers. These are derived from chemical bonds within molecules and between molecules.

The “Raman scattering signal(s) having a specific wavenumber” in this embodiment is derived from type b oxidized heme (hereinafter also referred to as oxidized heme b), which is a prosthetic group bonded to the CYP enzyme. Specifically, the specific wavenumber is preferably within the range of 300-600, 620-880, 920-1320, or 1320-1660 cm⁻¹. More preferably, the wavenumber is 1360-1380, or 1630-1640 cm⁻¹. Particularly preferably, the wavenumber is 1370 or 1636 cm⁻¹. In one specific embodiment, first, cells are irradiated with an excitation light and the generated Raman scattered light is dispersed using diffraction grating. Next, among the Raman spectra detected by the detection instrument, a Raman scattering signal (Raman scattering intensity) is obtained at a wavenumber of, for example, 1630-1640 cm⁻¹. A Raman scattering signal obtained at this wavenumber indicates that the CYP enzyme group has enzyme activity. Note that the bond between the CYP enzyme and heme b is a covalent bond or a non-covalent bond.

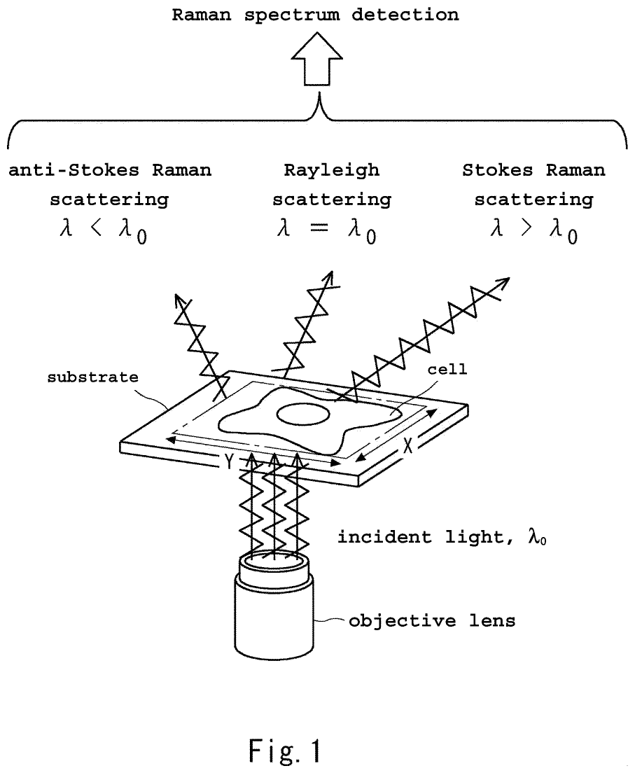

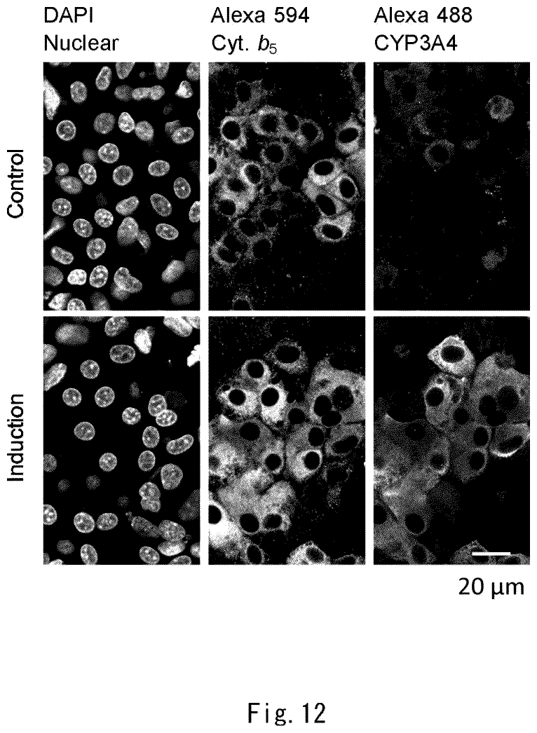

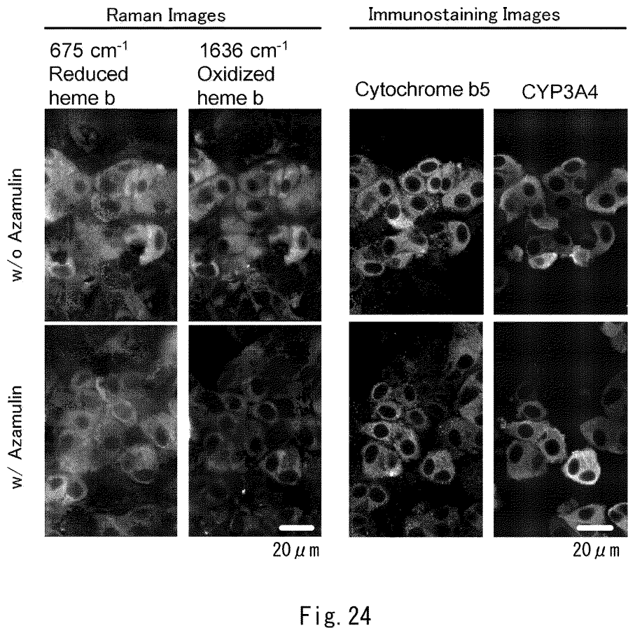

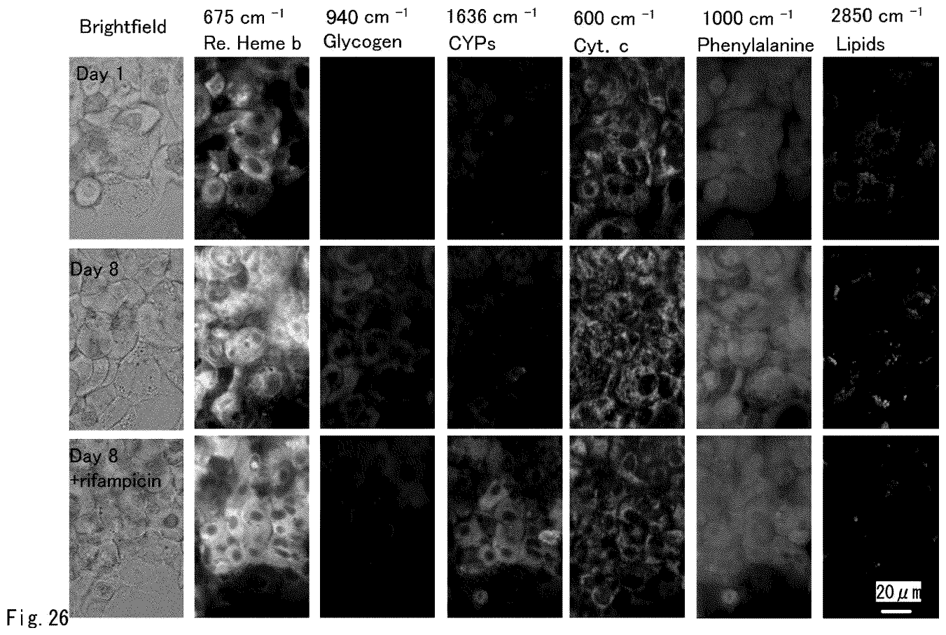

In this embodiment, cell shape, cell size, and intracellular distribution of intracellular components may be observed in the region where the enzyme activity of CYP enzyme group is evaluated along with the evaluation of enzyme activity of CYP enzyme group. As used herein, the “region where the enzyme activity of CYP enzyme group is evaluated” is, in one embodiment, a predetermined area containing cells spread on a substrate, as shown in FIG. 1, which is a flat plane of X μm width and Y μm length in the Figure. In FIG. 1, one cell is arranged in the region, but a plurality of cells may be arranged therein. The cell shape, cell size, and intracellular distribution of intracellular components can be observed using, for example, an optical measuring method. The optical measuring method refers to, for example, a method of generating images in bright field (FIG. 26) or fluorescence (FIG. 12, FIG. 24). By using an optical measuring method concurrently, localization of the cell group to be observed can be specified and the cell group to be analyzed can be identified (specifying the hepatocyte part), thus allowing for efficient measurement and highly sensitive signal extraction. Furthermore, since it is possible to identify individual cells (extraction of cell outlines), analyses such as quantification of the activity of each cell and the like can be performed. Furthermore, analysis using organelles and CYP enzyme activity distribution is also possible by fluorescently staining organelles and analyzing the obtained fluorescence images and Raman images.

In this embodiment, simultaneously with the evaluation of the enzyme activity of the CYP enzyme group, other molecules or the redox states thereof may be detected by further using other Raman scattering signals included in the Raman spectrum. A Raman scattering signal having a specific wavenumber indicating a molecule may be derived from any of reduced/oxidized heme b, reduced/oxidized heme c, glycogen, cytochrome c, phenylalanine, and lipid.

Other substances to be evaluated by the evaluation method of this embodiment are explained. In this embodiment, cytochrome c and glycogen can be used in addition to CYP as indicators of the metabolic ability of the liver. Metabolism is a reaction that produces the energy necessary to sustain life and synthesizes the necessary polymer compounds, and needs to be fully understood in the development of medical technology and drug discovery. As a method for measuring cytochrome c and glycogen, methods using a luminescence method or a fluorescence method have been widely used. Furthermore, in recent years, stain-free measurements using Raman spectroscopy have been attracting attention. Raman spectroscopy utilizes the fact that substances exhibit specific spontaneous Raman scattered light. Particularly, in the evaluation of hepatotoxicity and liver metabolism, drug metabolism by CYP, mitochondrial activity or toxicity using cytochrome c as an indicator, and intracellular energy production and storage using glycogen as an indicator are important parameters and are interrelated. Non Patent Literature 3 discloses Raman observation of cytochrome c in cells undergoing apoptosis.

Cytochrome c is a heme c protein present in cells or tissues. It is localized in mitochondria, one of the organelles, and is involved in apoptosis and energy production. Toxic effects on mitochondria cause drug hepatopathy, and the quantification and localization of cytochrome c are important factors. Cytochrome c contains type c heme as a prosthetic group. It emits a characteristic Raman signal that is different from type b heme contained in CYP and other proteins. That is, a Raman scattering signal derived from the redox state of cytochrome c can be used as one of the biomolecules other than CYP enzymes.

Additionally, type b heme proteins such as cytochrome b₅ can also be detected. Cytochrome b₅ plays an important role in supplying electrons in the catalytic cycle of CYP enzyme group.

In addition, detection of phenylalanine which indicates cytoplasm can be mentioned. The intensity of the Raman scattering signal of phenylalanine reflects the density of the cytoplasm in the region subjected to Raman observation.

The specific activity of CYP can be obtained by normalizing (dividing) the Raman scattering signals derived from CYP activity by the Raman scattering signals derived from these molecules. For example, when the Raman scattering signals of cytochrome c is used for normalization, the specific activity of CYP relative to mitochondria activity can be obtained. When the Raman scattering signals of cytochrome b is used, the specific activity of CYP relative to the catalyst activity can be obtained. When normalized using the Raman scattering signal of glycogen, the specific activity of CYP relative to sugar metabolism can be obtained.

In addition, cytochromes c, b, glycogen, and phenylalanine show specific distributions inside the cell, that are derived from organelles (cytochrome c: distributed in mitochondria, cytochrome b: distributed in endoplasmic reticulum, phenylalanine: distributed in the whole cytoplasm).

Therefore, quantification of cell status and drug response by utilizing the relationship between each cell function that can be measured from Raman scattering signals thereof and the shape and localization of organelles can be expected.

Here, differences in Raman scattering signals due to differences in heme structure is explained. It has been reported that reduced heme c has multiple characteristic Raman scattering signals represented by wavenumber 604 cm⁻¹, reduced heme b has those represented by wavenumber 675 cm⁻¹, reduced heme structure (common to b and c) has those represented by wavenumber 750 cm⁻¹, and oxidized heme structure (common to b and c) has those represented by wavenumber 1638 cm⁻¹ (Non Patent Literature 4). In addition, it has been reported that, when the CYP enzyme group with heme structure or CYP3A4 takes an oxidized structure, characteristic Raman scattering signals are found around wavenumbers 1368 cm⁻¹, 1371-1373 cm⁻¹, 1490 cm⁻¹, 1500 cm⁻¹, 1570 cm⁻¹, 1590 cm⁻¹, 1630 cm⁻¹, 1640 cm⁻¹. (Non Patent Literatures 5 to 12).

The wavenumber of Raman scattering signals derived from reduced/oxidized heme b may be in the range of 650-680 cm⁻¹, 900-1000 cm⁻¹, 1300-1373 cm⁻¹, 1490-1500 cm⁻¹, or 1570-1590 cm⁻¹ (Non Patent Literature 13).

The wavenumber of Raman scattering signals derived from reduced/oxidized heme c may be in the range of 590-640 cm⁻¹, 730-755 cm⁻¹, 1120-1130 cm⁻¹, 1310-1370 cm⁻¹, or 1580-1640 cm⁻¹.

Glycogen is known to be a polymer synthesized for temporary storage of excess glucose, and be synthesized mainly in the liver and skeletal muscles. It is an effective indicator of liver function and glucose metabolism. The wavenumber of Raman scattering signals derived from glycogen may be in the range of 440-580 cm⁻¹, 840-945 cm⁻¹, 1020-1660 cm⁻¹ (more preferably, 1020-1120 cm⁻¹), or 2900-2940 cm⁻¹.

The wavenumber of Raman scattering signals derived from lipid may be 1450 cm⁻¹ or 2850 cm⁻¹.

In the following, a method for detecting Raman scattering of CYP enzyme group and other substances in the cell, and a method for identifying Raman scattering signal with a specific wavenumber from the acquired Raman spectrum are explained with reference to FIG. 1 to FIG. 5.

FIG. 1 is a diagram explaining the principle of Raman scattering. As shown in FIG. 1, when a sample is irradiated with an excitation light of wavelength λ₀ such as a laser, scattering occurs in addition to reflection, refraction, absorption, and the like. Among the scattered light of wavelength λ, one that has the same wavelength as the incident light is called Rayleigh scattering. On the other hand, light whose frequency has decreased compared to the incident light, that is, whose wavelength has shifted to the longer wavelength side, is called Stokes Raman scattering (hereinafter Raman scattered light). A Raman spectrum is obtained by separating Raman scattered light generated from a sample, by diffraction grating and detecting same with a detection device such as a CCD image sensor. Then, Raman scattering signals are extracted from the acquired Raman spectrum.

As a sample to be irradiated with an excitation light, for example, tissue from living organism (in vivo), cultured cells (in vitro), immobilized tissue from living organism, and the like may also be used. Examples of the tissue from living organism include liver tissue, intestinal tissue (preferably small intestine tissue), kidney tissue, brain tissue, and cells therein, all of which are derived from human or animal.

Examples of the cultured cell include liver cells, small intestinal epithelial cells, bile duct epithelial cells, renal cells, nerve cells, glial cells, and the like. In one embodiment, the liver cell is a hepatocyte. In one embodiment, the cultured cell may be a primary cultured cell or an immortalized cell. In one embodiment, the primary cultured cell may be a primary hepatocyte derived from human (PHH). In one embodiment, the immortalized cell may be a cell line derived from human liver tumor. In one embodiment, the cultured cell is a cell differentiated from these. As the cell line derived from human liver tumor, for example, HepaRG (trademark, HPR116, BIOPREDIC International) cells may also be used. In addition, hepatocytes differentiated or dedifferentiated from HepaRG cells may also be used. In one embodiment, the cultured cell may be a cell differentiated from a stem cell. As the stem cell, ES cell, iPS cell, somatic stem cell (e.g., neural stem cell, hepatic stem cell, epithelial stem cell, etc.), and the like can be mentioned, but are not limited to these. In one embodiment, the stem cell may be a pluripotent stem cell (i.e., ES cell or iPS cell). In one embodiment of the evaluation method of the present invention, the cultured cell may be a liver cell obtained by inducing differentiation of human iPS cell (Hepatocyte-Like Cell, HLC)).

Raman scattering of purified proteins of CYP enzymes may be observed. The “purified protein” is a CYP extracted from cells or a synthesized CYP, from which contaminants have been completely or partially removed. Synthesized CYP is a gene recombinant protein prepared microbiologically. The “environment simulating an intracellular environment” is an in vitro reaction system containing an intracellular substance other than CYP enzyme.

Cultured cells are diluted with a medium and seeded on a quartz substrate dish for Raman observation. The quartz substrate dish for Raman observation is a plastic dish with a quartz substrate adhered on the bottom surface inside the dish.

When cultured cells are seeded, cloning cylinders may be used to seed the cells only on quartz substrates. In one example, when HepaRG (trademark) cells are used, cells cultured for 3 days at 37° C. in a 5% CO₂ atmosphere may be used as a sample.

As a sample other than those mentioned above, when Raman scattered light is detected outside cells, a Raman scattering target sample can be, but is not limited to, a solution sample extracted from a tissue and containing contaminants, for example, microsome. In the present specification, “outside a cell” is used to mean “in vitro” unless otherwise specified, but may be used to mean “extra-cellular” in some contexts.

The CYP enzyme group whose enzyme activity is evaluated in this embodiment may be induced intracellularly using an inducing agent. Rifampicin, which is one type of inducing agent of CYP enzyme group, mainly induces the expression of CYP3A4 among the CYP enzyme group. As mentioned above, this inducing agent can also induce the expression of CYP2B6, CYP2C9, and the like.

In this embodiment, Raman microscope (Non Patent Literature 3) may also be used to specify Raman scattering signals from Raman spectrum having a specific wavenumber. In this microscope, a laser beam is condensed into a straight line with a predetermined length (X μm, hereinafter also referred to as a line) to excite Raman signals of a sample. Note that the laser light may be condensed into a point to excite the Raman signal.

For example, a water immersion objective lens may also be used for excitation and detection of Raman scattering. The Raman scattered light collected by the objective lens passes through a dichroic mirror and an edge filter that transmits long wavelengths, and then is condensed and imaged on an entrance slit of a spectroscope. The Raman scattering signal imaged on the slit is then separated by diffraction grating inside the spectroscope and detected by a CCD image sensor. For example, a cooled CCD image sensor may be used as the CCD image sensor.

Raman scattering signals scattered from everywhere on the condensed line are detected by different pixels on the CCD image sensor. It is preferable to determine the laser irradiation time so that these Raman scattering signals can be measured simultaneously and independently. The laser irradiation time is, for example, 0.001 second to 3 minutes. It is preferably 0.01 second to 30 seconds. More preferably, it is 0.1 second to 15 seconds. Particularly preferably, it is 1 second to 10 seconds. When the light is condensed in a straight line, each Raman scattering signal is detected simultaneously in a straight line region (line region) of a predetermined length where the light is condensed. Laser irradiation and Raman scattering signal detection are repeated while shifting the light condensing position in a direction perpendicular to the direction of the condensed line. For example, as shown in FIG. 1, a Raman image is acquired in a rectangular area of X μm×Y μm on the substrate.

When the light is condensed into a point, a Raman image of any area is obtained by repeatedly detecting the Raman scattering signal while shifting to any distance in the X-Y direction. As a result, a Raman image is finally obtained from an area of X μm in width and Y μm in length. The X and Y directions may be exchanged.

As the excitation light, for example, it is preferable to use a laser beam with a wavelength of 406 nm to 561 nm, particularly a laser beam with a wavelength of 532 nm. The present inventors took note of resonance Raman scattering. In resonance Raman scattering, by exciting molecules with the absorption bands that the molecules have, vibrations derived from the absorption bands can be selectively measured due to the resonance effect. Heme b and heme c have an absorption band called R/a band around 520 to 560 nm. Therefore, when a laser beam with a wavelength of 532 nm is used as the excitation light, the Raman scattered light can be significantly increased due to the resonance effect. The above-mentioned heme protein also has an absorption band called Soret band near 380 to 460 nm, and therefore, Raman scattering may be excited using a laser beam with a wavelength of 488, 561, 556, 543, 526, 523, 520, 515, 501, 450, 406 nm, or the like. The Raman scattered light detected by the Raman scattering method of this embodiment is due to resonance Raman scattering. In this embodiment, Raman scattered light due to non-resonant Raman scattering may also be detected.

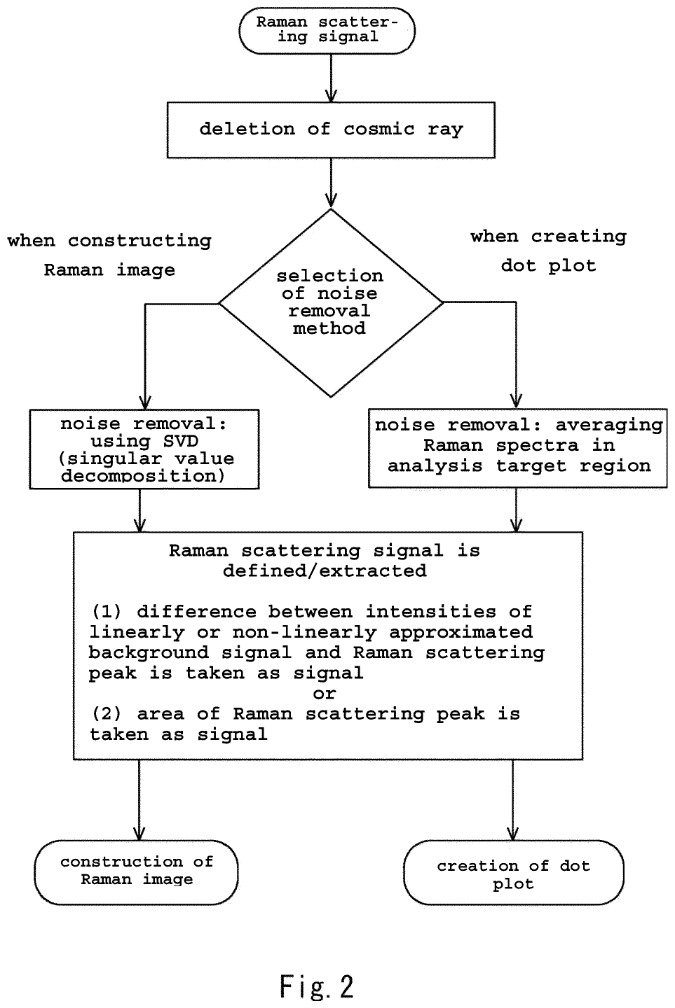

FIG. 2 is a flowchart illustrating a method for extracting Raman scattering signals. First, cosmic rays and offset signals of the CCD image sensor, which are noises included in the measured Raman scattering signals, are removed. The offset signal is a signal for adjusting the offset of the CCD image sensor. Subsequently, a treatment is performed to reduce the influence of noise that could not be completely removed above. As a method for removing noises, either a method using a singular value decomposition (SVD) method or a method of averaging Raman spectra for any each analysis target region can be used. When constructing a Raman image, singular value decomposition can be used. Furthermore, when creating a dot plot, a method of averaging Raman spectra for any each analysis target region can be used.

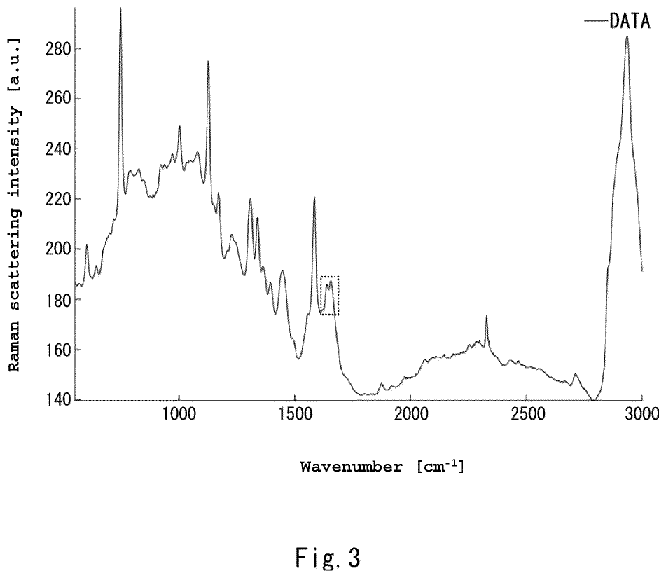

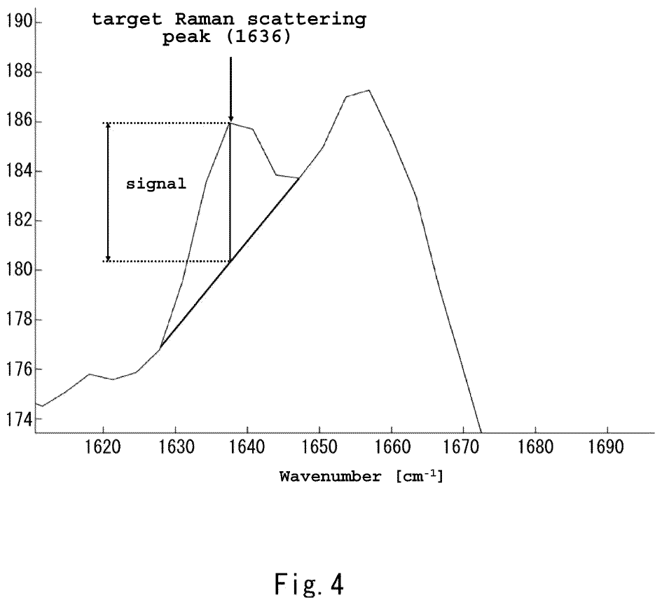

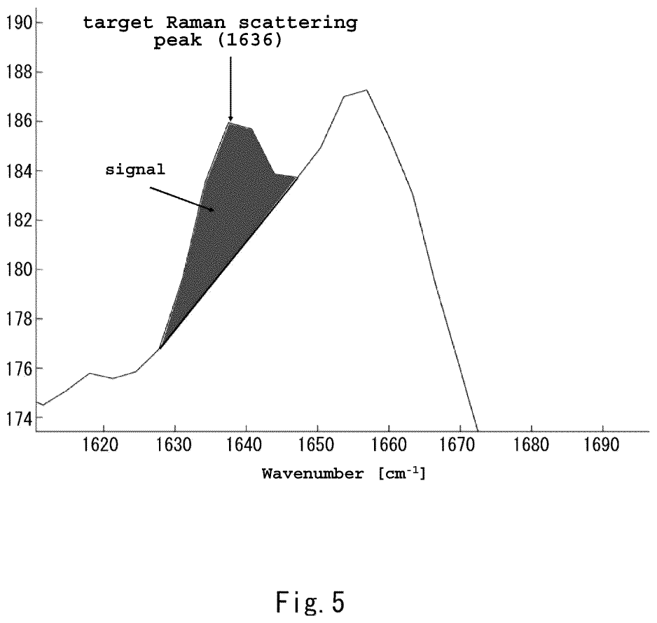

After removal of the noises contained in the Raman scattering signal, Raman scattering signal of the analysis target is extracted. As used herein, Raman scattering signal of the analysis target refers to the Raman scattering signals (Raman scattering intensity) at a wavenumber of 1636 cm⁻¹ when detecting the enzyme activity of the CYP enzyme group. Furthermore, the “extraction” of Raman scattering signal of the analysis target means defining any Raman scattering intensity as Raman scattering signal of the analysis target by using a predetermined method. As a method for extracting Raman scattering signal of the analysis target, the first method or the second method described below can be used. The first method is explained using FIG. 3 and FIG. 4, and the second method is explained using FIG. 3 and FIG. 5.

FIG. 3 shows the Raman spectrum after noise removal. As shown in FIG. 3, Raman spectrum (Raman scattering signal) is represented by the Raman scattering intensity (Raman scattering signal intensity) on the vertical axis and the Raman shift (wavenumber) on the horizontal axis. The Raman shift (wavenumber) on the horizontal axis shows the difference in wavenumber between incident light from excitation light such as laser or the like, and Raman scattered light.

FIG. 4 is an enlarged view of the area indicated by the broken line in FIG. 3. Using this Figure, a method for extracting Raman scattering signal of the analysis target, which uses the first method, is described. The first method is suitable when a peak of the Raman scattering intensity of a Raman scattering signal derived from another living body exists in the vicinity of Raman scattering signal of the analysis target.

In the first method, the difference between the intensity of linearly or non-linearly approximated background signal and the peak of the Raman scattering intensity is defined as Raman scattering signal of the analysis target. More specifically, first, the peak of Raman scattering intensity at specific wavenumber and a wave form containing the peak are specified. The intersection of a line connecting both ends of the base of the aforementioned peak, that is, a straight line or a curved line, and a straight line drawn perpendicularly from the peak vertex to the wave number axis is set as the point of origin, and then the height from the aforementioned point of origin to the aforementioned peak vertex is calculated as Raman scattering signal of the analysis target. In the present specification, the “line connecting both ends of the base” is not particularly limited as long as it can define the region of the peak. The line may be an n-dimensional curve. When n=1, the line is a straight line, and when n>1, the line is a so-called curved line.

FIG. 5 is an enlarged view of the area indicated by the broken line in FIG. 3. Using this Figure, a method for extracting the analysis target Raman scattering signal, which uses the second method, is described. The second method is suitable when Raman scattering signal of the analysis target has a sufficient signal-to-noise ratio (SN ratio) with respect to the background signal, as well as a peak of the Raman scattering intensity derived from another living body does not exist in the vicinity.

In the second method, first, the peak of Raman scattering intensity at specific wavenumber and a wave form containing the peak are specified. For the peak of the Raman scattering intensity of the analysis target, the area of the region surrounded by the line connecting both ends of the base of the aforementioned peak, that is, a straight line or a curved line, and the waveform of the Raman scattering signal is calculated and defined as Raman scattering signal of the analysis target. In the present specification, the “line connecting both ends of the base” is not particularly limited as long as it can define the region of the peak. The line may be an n-dimensional curve. When n=1, the line is a straight line, and when n>1, the line is a so-called curved line.

In addition to the first method and the second method, it is also possible to extract Raman scattering signal of the analysis target using another method according to the required accuracy of quantitativeness. For example, the difference between the intensity of the peak of Raman scattering signal of the analysis target and the intensity at the base of the peak is defined as Raman signal of the analysis target. The base of the peak refers to a convex point on the y-axis minus direction in the vicinity of the peak of Raman scattering signal of the analysis target.

By the method described above, Raman scattering signal of the analysis target is defined at a wavenumber of 1320 to 1660 cm⁻¹, preferably 1636 cm⁻¹. The Raman scattering signal (Raman scattering intensity) increases in a cell, enzyme activity of CYP enzyme group in the cell increases. Using Raman spectroscopy, Raman signals of biomolecules relating to metabolic ability can be detected from living cells under a microscope. In addition, Raman signals from biomolecules that indicate the state and structure of cells can also be detected at the same time. Furthermore, the intracellular distribution of each detected biomolecule can be understood with spatial resolution at the organelle level, with a spatial resolution of 200 nm in one example. Therefore, when drug response of cells in metabolism and energy production are examined, comprehensive examination is possible combined with the behavior of biomolecules localized in other organelles. Observation of Raman signals at 1636 cm⁻¹ can contribute not only to the measurement of the activity of CYP enzyme group but also to the elucidation of the mechanisms of cell functions thereof. Furthermore, at least one of the type, differentiation degree, and maturity of the cell or tissue can also be evaluated by detecting indicators of liver function such as glucose metabolism and mitochondrial activity/toxicity, by using the Raman scattering signals of glycogen and cytochrome c extracted from the Raman spectrum.

Based on the aforementioned facts, the present invention also provides a method for evaluating a metabolic ability of hepatocyte, including the following steps (hereinafter sometimes referred to as “the method for evaluating metabolic ability of hepatocyte of the present invention”):
a step of irradiating an excitation light on hepatocytes and obtaining a Raman spectrum using a photodetector, and
a step of detecting Raman signals of biomolecules relating to metabolic ability of hepatocyte from the aforementioned Raman spectrum.

In the method for evaluating metabolic ability of hepatocyte of the present invention, the “biomolecule relating to metabolic ability of hepatocyte” is not particularly limited as long as it is a biomolecule related to the metabolic ability of hepatocytes. It may be, for example, at least one selected from the group consisting of CYP enzyme group, glycogen, cytochrome b₅, cytochrome c, lipid, and phenylalanine. As the wavenumber of the Raman scattering signal that can detect these biomolecules, a wavenumber known per se may be used. Specific examples include, but are not limited to, the following wavenumbers used in the following Examples.

### EXAMPLE

The above-mentioned embodiments are described in more detail with reference to Examples 1 to 7. Experiment conditions and the like that are common to these Examples are explained in (1) to (8) below. In the following Examples, CYP3A4 and other CYP enzyme group in human liver tumor-derived cells are detected. As mentioned above, the detection method according to the present invention is a method utilizing the redox state derived from the characteristic molecular structure of the CYP enzyme group. Furthermore, as mentioned above, the redox state of the CYP enzyme group is related to the mechanism of the metabolism of the CYP enzyme group. Therefore, the data obtained for CYP3A4 in the following Examples demonstrate that the enzyme activity of metabolism not only by human CYP3A4 but also by CYPs from all kinds of organisms can be detected by the detection method of the present invention.

### (1) Seeding and Culture of Hepatocytes

In Examples, cultured cells were used. More specifically, frozen HepaRG cells (HPR116, BIOPREDIC International) differentiated into liver cells and bile duct epithelial cells were used. Frozen HepaRG cells were thawed in a water bath at 37° C. Then, the thawed HepaRG cells were diluted with a medium (MIL600, ADD670, BIOPREDIC International). Then, the diluted HepaRG cells were seeded on a quartz substrate dish for Raman observation (SF-S-D12, Fine Plus International, Japan).

Seeding is more specifically explained. The “quartz substrate dish for Raman observation” used for seeding is a plastic dish with a quartz substrate (thickness 0.15 mm, diameter 12 mm) adhered on the bottom surface inside the dish.

In order to seed HepaRG cells only on the quartz substrate of the dish, first, a cylindrical cloning cylinder (outer diameter 10 mm, inner diameter: 8 mm, 1980005, Hilgenberg GmbH) was placed on the quartz substrate. 1.2×10⁵ cells were seeded inside the cloning cylinder. Note that the cloning cylinder is placed on the quartz substrate. In other words, the cloning cylinder is not adhered or fixed onto the quartz substrate.

24 hours after seeding, HepaRG cells adhere to the quartz substrate. At the time point when the seeded HepaRG cells adhered to the quartz substrate, the cloning cylinder was removed from the quartz substrate. When the cloning cylinder was removed, the medium was replaced with a fresh medium (MIL600, ADD670, BIOPREDIC International). Successively, HepaRG cells were cultured in the medium. Culture was performed at 37° C. under a 5% CO₂ atmosphere for 3 days.

(2) Induction of CYP Enzyme Group Expressed in Liver Cells or Inhibition of CYP3A Then, using the HepaRG cell population cultured according to the description of the above-mentioned (1), induction or inhibition of CYP enzyme group was performed.

Induction of CYP enzyme group is explained. First, the medium of the aforementioned (1) HepaRG cell group was replaced with a medium containing an inducing agent for CYP enzyme group. The HepaRG cell group was cultured in a replaced medium containing an inducing agent explained below at 37° C. under a 5% CO₂ atmosphere for 48 hr to induce CYP enzyme group.

The media used for the induction of CYP enzyme group were MIL600, ADD650, BIOPREDIC International. An inducing agent for the CYP enzyme group added to the media was any of the following four kinds: Rifampicin (189-01001), Fujifilm Wako, Japan; Phenytoin (16612082) Fujifilm Wako, Japan; Dexamethasone (04718863), Fujifilm Wako, Japan; and Carbamazepine (034-23701) Fujifilm Wako, Japan. The concentration of each inducing agent contained in each medium used in each Example was as described in each Example. These inducing agents promote transcription of CYP genes.

Control cells free of induction of CYP enzyme group (hereinafter also referred to as non-induced cell group) were also cultured. For the non-induced cell group, the medium of the HepaRG cell group in (1) was replaced with the aforementioned medium containing no aforementioned inducing agent (MIL600, ADD650, BIOPREDIC International). The cells were cultured at 37° C. under a 5% CO₂ atmosphere for 48 hr.

Inhibition of the CYP enzyme group is now explained. The HepaRG cell group used to inhibit CYP3A, which is a type of CYP enzyme group, is cells in which induction of the aforementioned CYP enzyme group was performed. To be specific, the medium of the HepaRG cells of (1) was replaced with a medium containing Rifampicin which was one type of the aforementioned inducing agent, and the cells were cultured at 37° C. under a 5% CO₂ atmosphere for 48 hr to induce CYP enzyme group and the cells were obtained. The cells were used for the inhibition of CYP3A.

The media used for the inhibition of CYP enzyme group were MIL600, ADD650, BIOPREDIC International. An inhibitor added to the media was Azamulin (18748, Cay chemical). The concentration of Azamulin contained in the medium used in each Example was as described in each Example. The medium used for inducing CYP enzyme group was replaced with a medium containing Azamulin as the inhibitor. HepaRG cell group was cultured at 37° C. under a 5% CO₂ atmosphere for 5 min in the replaced medium containing Azamulin to inhibit CYP3A which is one type of CYP enzyme group.

### (3) Detection of CYP3A4 Activity in Cell Group Using Luminescence Method

In order to confirm the enzyme activity of CYP3A4, which is particularly expressed in liver cells among the CYP enzymes in the cell group subjected to Raman observation, a test using a luminescence method (hereinafter a luminescence test) was performed in parallel with the Raman observation. The cell is group used in the luminescence test was a cell group separated from the cultured cells used in the below-mentioned Raman observation in (5). That is, the cell group cultured under the same conditions and at the same time as the cultured cells used in Raman observation was used. CYP luminescence test was performed using a commercially available luminescence assay kit (P450-Glo-CYP3A4-Assay-and-Screening-System) according to the provided protocol.

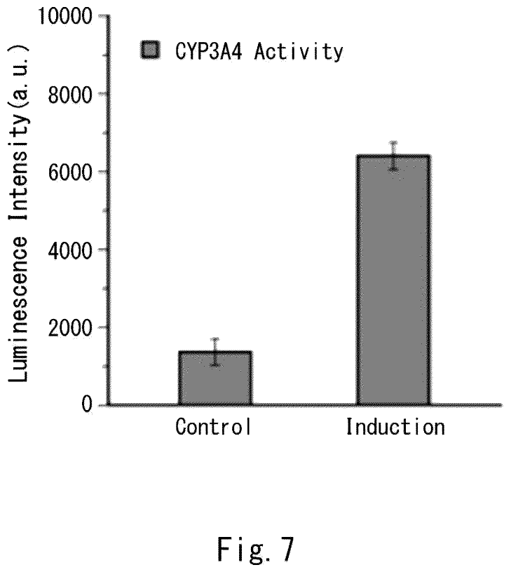

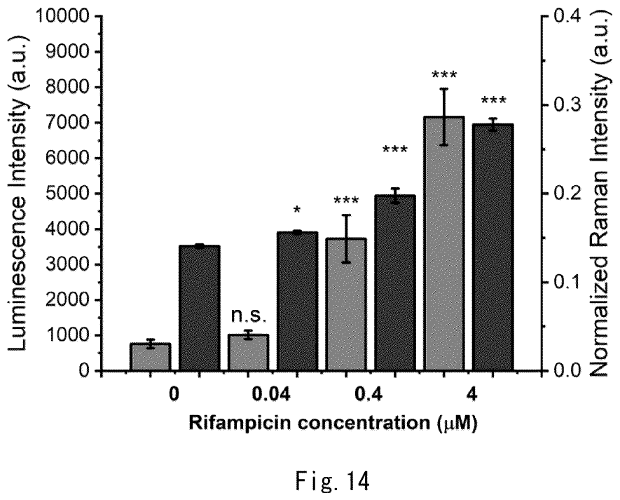

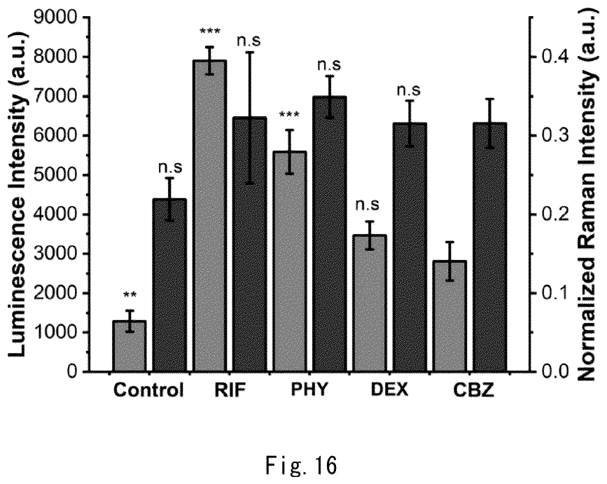

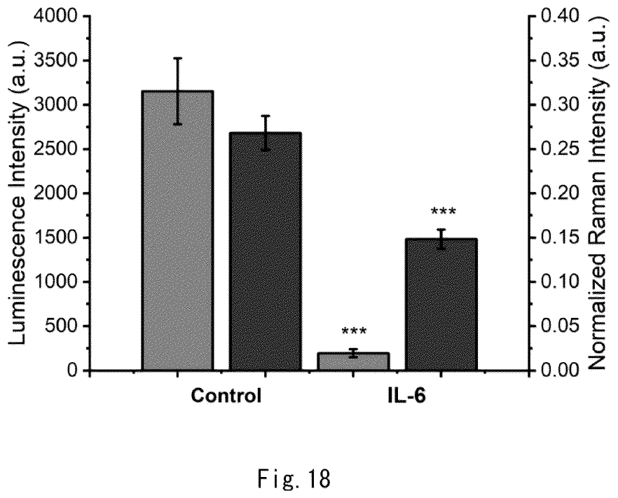

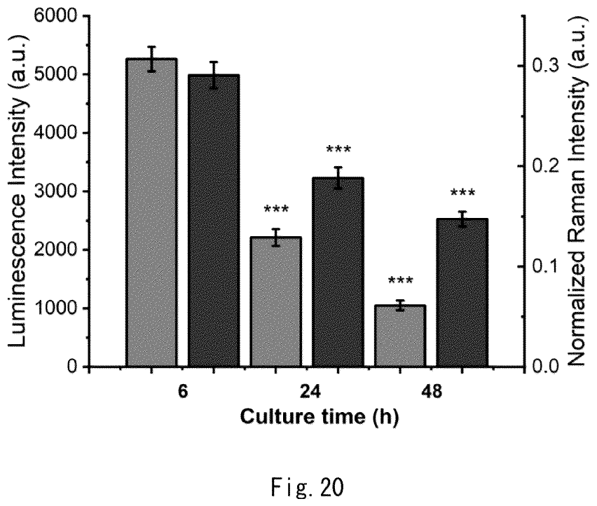

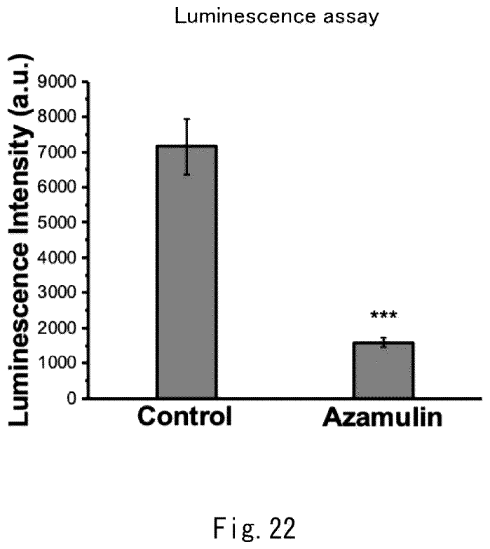

When the Luciferin-IPA substrate is converted to luciferin by CYP3A4, it becomes a substrate for luciferase, producing luminescence proportional to the activity of CYP3A4. The medium of the cell group was replaced with a substrate-containing medium containing Luciferin-IPA substrate (V9002, Promega, Germany) at a concentration of 3 μM and incubated at 37° C. under a 5% CO₂ atmosphere for 1 hr. Here, the Luciferin-IPA substrate is a substrate specific to CYP3A4. Luciferin-IPA substrate, which is a luciferin precursor, is converted to luciferin by the catalytic action of the CYP3A4 enzyme. After incubation, the substrate-containing medium was collected and 50 μl was dispensed into a 96-well white flat bottom plate (Corning). Luciferase (50 μl) was added dropwise to the dispensed substrate-containing medium, and luminescence was caused using the luciferin-luciferase reaction. Luminescence was detected using a micro-plate reader (Synergy HTX, BioTek). The CYP3A4 activity detected by the present luminescence method is shown in FIG. 7, FIG. 14, FIG. 16, FIG. 18, FIG. 20, and FIG. 22.

### (4) Detection of CYP in Cell Group by Using Western Blotting

Using Western blotting, CYP3A4 protein was detected from the cell group in which the CYP enzyme group was induced. In Western blotting, the cell group was used which was cultured by inducing the CYP enzyme group by using Rifampicin as the inducing agent according to the method described in the aforementioned (2). The CYP-induced cell group was washed twice with cooled PBS and dissolved in a cell lysis solution. Lysis of the cells was performed in cell lysis solution RIPA Buffer containing protease inhibitor cocktail (1-100 dilution, Cat. No. P8340, Sigma Aldrich) for 30 min while cooling on ice. A cell lysis solution containing the lysed cell group was collected using a cell scraper. The collected cell lysis solution was centrifuged at 4° C. (12000 g, 20 min), and the supernatant was collected to remove unnecessary precipitated cell debris.

The concentration of the protein contained in the supernatant of the cell lysis solution was measured using a BCA protein assay kit (Cat. No. 23227, ThermoFisher Scientific). The cell lysis solution was added to the wells of 12% SDS-PAGE gels (Cat. No. 4568043, BioRad) and subjected to electrophoresis. A low fluorescence polyvinylidene fluoride (PVDF) membrane was closely adhered to the gel after SDS-PAGE, and the gel was transferred. The membrane after transfer was blocked using a TBST (0.1% Tween-20 in TBS) solution containing 5% ECL blocking agent (Cat. No. RPN2125, GE Healthcare) for 1 hr at room temperature while stirring with a stirrer. The membrane was then washed twice with TBST solution.

Successively, the primary antibody reaction and the secondary antibody reaction were performed. The blocked membrane was soaked in a blocking buffer containing a primary antibody, mouse anti-CYP3A4 (1:2000), rabbit anti-cytochrome b₅ (1:1000), and rabbit anti-β-actin (1:1000 dilution, Cat. No. 4970, Cellsignaling Technology), and reacted overnight. Then, the membrane was soaked in a blocking buffer containing a secondary antibody, horseradish peroxidase-conjugated anti-mouse or anti-rabbit secondary antibodies (1:10000), and reacted at room temperature for 1 hr. The membrane was then soaked in TBST buffer and washed three times.

CYP3A4 protein was detected using the ECL detection system (RPN2232, GE Healthcare) and ChemiDOC MP imaging system (Bio-Rad).

### (5) Raman Observation of Cell Group by Using Raman Spectroscopy

A Raman image of the cell group cultured using the above-mentioned method (1) or (2) was constructed by quantitatively measuring the Raman signal using Raman spectroscopy. Here, the “observation” in the present specification includes both detecting a Raman scattering signal of a cell group and acquiring and observing a Raman image of a cell group. Depending on the Example, a cell group to be observed may be obtained through other step after culturing by the method of (1). Details are explained in each Example.

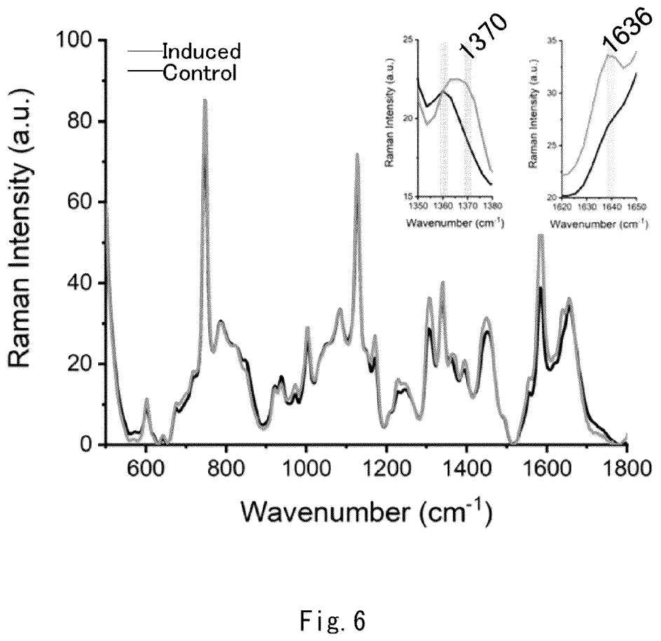

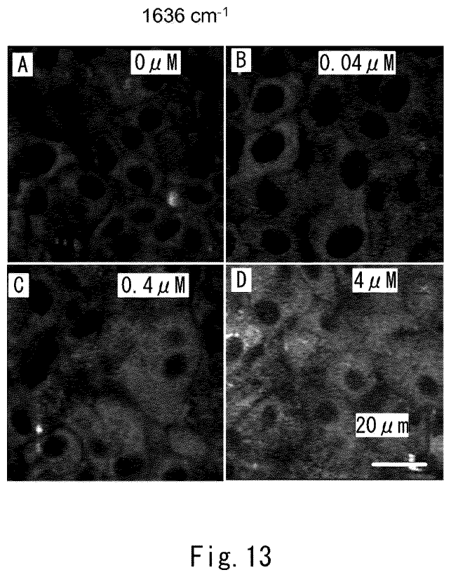

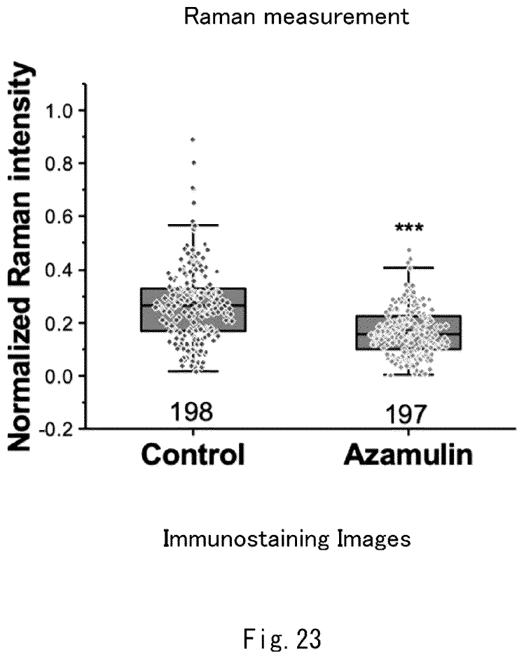

The culture medium of the cultured cell group was removed, and the cell group was washed twice with PBS. An observation solution (Live Cell Imaging Solution (A14291DJ, Thermo Fisher Scientific)) was added to the washed cell group. The thus-treated cell group was subjected to Raman observation using the Raman microscope (Non Patent Literature 3) described using FIG. 1 (FIG. 6, FIG. 9 to 11, FIG. 13 to FIG. 21, FIG. 23 to FIG. 26).

A 40× water immersion objective lens (CFI Apochromat Lambda S 40XC WI) equipped in a Raman microscope was used to excite the Raman scattered light of the cell group and to detect the Raman scattering signal. The excitation light of the Raman scattered light of the cell group was irradiated by condensing a laser beam with a wavelength of 532 nm into a straight line with a predetermined length (hereinafter referred to as a line) using the water immersion objective lens.

The flow of detection of Raman scattered light of a cell group that was generated by excitation with excitation light is explained. The Raman scattered light collected by water immersion objective lens passes through a dichroic mirror and an edge filter that transmits long wavelengths, and then is condensed and imaged on an entrance slit of a spectroscope. The Raman scattering signal imaged on the slit is then separated by diffraction grating inside the spectroscope and detected by a cooling-type CCD image sensor (PIXIS 400B, Princeton Instruments) used as a photodetector. Raman scattering signals scattered from everywhere on the condensed line can be measured simultaneously and independently by different pixels on the CCD. The laser irradiation time for one time was set to 5 seconds. Respective Raman signals were simultaneously detected in an about 133.3 μm region on the direction of the condensed line. The laser irradiation and Raman signal detection were repeatedly scanned while shifting the light condensing position by scanning pitch Z nm in a direction perpendicular to the direction of the condensed line. That is, a laser was scanned in a rectangular region of 133.3 μm× Y μm to detect Raman scattering signals of the cell group. The density of laser energy on the surface of the cell group when scanning the laser was 3 mW/μm². The value of Y corresponding to the width of the observation area and the value of the scanning pitch Z in the direction parallel to Y vary depending on each Example. Two types of diffraction gratings with different numbers of gratings were used to detect Raman signals. Here, the “detection” of Raman scattering signals includes both quantitative measurement of the Raman scattering signal and construction of a Raman image. A diffraction grating with 1200 lines (1200 L/mm, BLZ500 nm) was used for quantitative measurement of Raman scattering signals. On the other hand, a diffraction grating with 600 lines (600 L/mm, BLZ500 nm) was used to construct the Raman image. This time, different diffraction gratings were used depending on the purpose of each experiment; however, it is not necessarily required to use them differently as described above. In other Examples, diffraction gratings with 600 and 1200 lines can be used for quantitative measurement of Raman scattering signals and construction of Raman images, respectively.

### (6) Noise Processing of Raman Scattering Signals

The Raman scattering signal measured as a Raman spectrum was analyzed using the calculation software Matlab (Math works) according to the method described in Non Patent Literature 14. After removing the cosmic ray contained in the measured Raman spectrum and the offset signal of the photodetector, noise was removed using singular value decomposition (SVD). After that, a signal intensity distribution of any Raman shift (wavenumber) was constructed and a Raman image was obtained. For quantitative measurement of Raman signals, noise removal by SVD was not performed, and data was used that removed cosmic rays and camera offset. Where necessary, the average of clear spectra, hereinafter referred to as average spectrum, was calculated from multiple Raman spectra. The plurality of Raman spectra may be Raman spectra from a region where target cells gather or from adjacent regions.

### (7) Extraction of Raman Scattering Signals and Construction of Raman Images

A clear spectrum was obtained by removing noise from the obtained Raman spectrum, by the method described in the above-mentioned (6). The Raman scattering signal of analysis target is extracted from the obtained spectrum. The extraction method varies depending on the situation, such as the intensity of the target Raman scattering signal and whether Raman scattering signals derived from other biomolecules exist in the vicinity. In this Example, the target Raman scattering signal was extracted from the spectrum by using the two methods described below, and used for quantification.

First, from the noise-processed spectrum, the peak and waveform containing the peak of Raman scattering signal of the analysis target, which is considered to derive from the CYP metabolism enzyme group, are identified. As one method, the Raman scattering signal intensity was calculated by defining the area of the region surrounded by the straight line or curve connecting both ends of the peak base and the waveform of the Raman scattering signal as the Raman scattering signal intensity. In another method, a straight line or a curved line connecting both ends of the base of the peak, and a straight line drawn perpendicularly from the peak vertex to the wavenumber axis is set as the point of origin, and then the height from the aforementioned point of origin to the aforementioned peak vertex was defined as Raman scattering signal intensity and the Raman scattering signal intensity was calculated. The signal intensity of the Raman scattering signal extracted by the method described using FIG. 4 was used to create each box-whisker plot in this Example. Using the above-mentioned two methods, the correlation between the analysis target Raman scattering signal and CYP activity was investigated.

### (8) Localization Observation of CYP3A4 and Cytochrome b₅ in Cell Group by Using Immunostaining

Using immunostaining, it was confirmed that the Raman signal with a wavenumber of 1636 cm⁻¹ was derived from the CYP enzyme group. Specifically, immunostaining was performed for CYP3A4 and cytochrome b₅ protein, which is a type B oxidized heme protein similar to the CYP enzyme group. Cytochrome b₅, like the CYP enzymes, is an enzyme involved in drug metabolism. The cell group used for immunostaining was the cell group after being subjected to Raman observation.

Immediately after Raman observation, the cell group was fixed with 4% paraformaldehyde for 20 min at room temperature. A permeation treatment of cell membrane was performed using a solution in which Triton-X-100 was diluted to 0.1% with PBS (phosphate buffered saline). After washing, a blocking treatment was performed for 1 hr at room temperature using 4% bovine serum albumin (A2153, Sigma Aldrich) to suppress non-specific staining. As primary antibody, 1% BSA blocking buffer containing anti-CYP3A4 (1:1000 dilution, SAB5300118, Sigma Aldrich) and rabbit anti-human cytochrome b₅ (1:500 dilution, ab69801, abcam) was added dropwise, and the mixture was left standing overnight under 4° C. The next day, after washing the cell groups three times with PBS solution, a blocking buffer containing Alexa Fluor 488 goat anti-mouse antibody (10 g/ml, A11001, Invitrogen) and Alexa Fluor 594 goat anti-rabbit antibody (10 g/ml, A11012, Invitrogen) was added dropwise as secondary antibody, and the mixture was reacted at room temperature for 1 hr. Staining was performed after washing three times with PBS solution. For staining, DAPI (D1306, Invitrogen) was added at a concentration of 1M and the mixture was reacted at room temperature to stain the cell nucleus. Finally, the cell group was washed twice with PBS and stored at 4° C. in the dark until fluorescence observation. Fluorescence observation was performed using a laser scanning confocal microscope (CLSM, Nikon, Japan) to observe the same area as the Raman observation region. The area for Raman observation is marked in advance using a permanent marker. Based on the labels, the regions for Raman observation and fluorescence observation were determined. The fluorescent images obtained by the aforementioned method are shown in FIG. 12 and FIG. 24.

In the following, Examples 1 to 7 are described in detail using FIG. 6 to FIG. 26.

### Example 1

### <Detection of Raman Spectrum Caused by Induction of CYP Enzyme Group in Human Liver Cells (FIG. 6)>

In this Example, the expression of CYP enzyme group in HepaRG cells was induced using Rifampicin, an inducing agent of CYP enzyme group, and detection of the Raman scattering signals caused by the induction of CYP enzyme group was attempted. Rifampicin mainly induces the expression of CYP3A4 in the CYP enzyme group. This inducing agent additionally induces the expression of CYP2B6, CYP2C8, CYP2C9, and CYP2C19 (Non Patent Literature 15). During rifampicin induction, changes in the expression of the whole CYP enzyme group, including the above-mentioned CYPs, are controlled by increase or decrease in the expression level of CYP3A4. The reason therefor is that Rifampicin mainly induces the expression of CYP3A4 in the CYP enzyme group, and that CYP3A4 accounts for one-third of all the types of the CYP enzyme group expressed in the liver (Non Patent Literature 2).

In this Example, Raman observation, luminescence test, and Western blotting were performed in parallel on cultured cells. The luminescence test was performed to confirm the enzyme activity of the CYP enzyme group, especially CYP3A4, in the cell group to be subjected to Raman observation. Western blotting was performed in order to detect CYP3A4 protein from the cell group.

According to the procedures described in the above-mentioned (1) and (2), seeding and culture of human hepatocytes (HepaRG cells) and induction of CYP enzyme group were performed. The inducing agent (Rifampicin) for CYP enzyme group was added at a concentration of 4 μM. As a control for the cell group in which the CYP enzyme group was induced, a non-induced cell group was cultured using a medium free of an inducing agent for CYP enzyme group (MIL600, ADD650, BIOPREDIC International).

After 6 days of culture after seeding, HepaRG cells aggregate to form distinct cell populations, with hepatocytes and bile duct epithelial cells coming together separately. On the quartz substrate on which the cells are seeded, the areas where hepatocytes and bile duct epithelial cells form cell group can be respectively identified by bright field microscope observation. Here, it is known that the CYP enzyme group is expressed only in hepatocytes. Therefore, the target of Raman observation in this Example is hepatocyte.

In order to perform Raman observation and luminescence test in parallel, the cell groups for Raman observation and luminescence test were separated from the cell group of hepatocytes that underwent induction of the CYP enzyme group. In addition, a cell group of hepatocytes free of induction of the CYP enzyme group was also separated for each of Raman observation and luminescence test.

For Raman observation, according to the procedure described in the above-mentioned (5), a rectangular region of 133.3×84 μm was laser scanned at a scanning pitch of 333 nm. The obtained Raman spectra were analyzed according to the procedures described in the above-mentioned (5) to (7). In addition, in parallel with the Raman observation, a luminescence test was performed according to the procedure described in the above-mentioned (3). The activity of CYP3A4 expressed in the cell group that underwent induction of the CYP enzyme group was measured by the luminescence test.

Further, Western blotting was performed according to the procedure described in (4) to detect CYP3A4 protein.

First, the results of Raman observation are explained with reference to FIG. 6.

FIG. 6 shows the Raman spectrum of hepatocytes with the CYP enzyme group induced therein. For observation of the cell groups with or without induction of CYP enzyme group, the average spectrum of the region where hepatocytes assemble was calculated and displayed in a plot. As shown in FIG. 6, a thin gray line (Induced) shows the spectrum of the hepatocyte cell group in which the CYP enzyme group was induced by Rifampicin. A dark gray line (Control) shows the spectrum of the cell group of hepatocytes in which the CYP enzyme group was not induced.

From the average spectrum in FIG. 6, peaks and waveforms at wavenumbers 1370 and 1636 cm⁻¹ were identified. At wavenumbers 1370 and 1636 cm⁻¹, the signal intensity was compared between the cell group of hepatocytes in which the CYP enzyme group was induced and the cell group of hepatocytes in which the CYP enzyme group was not induced. It could be confirmed that Raman signals at wavenumbers of 1370 and 1636 cm⁻¹ increased in the cell group of hepatocytes in which the CYP enzyme group was induced using Rifampicin compared with the cell group in which the CYP enzyme group was not induced.

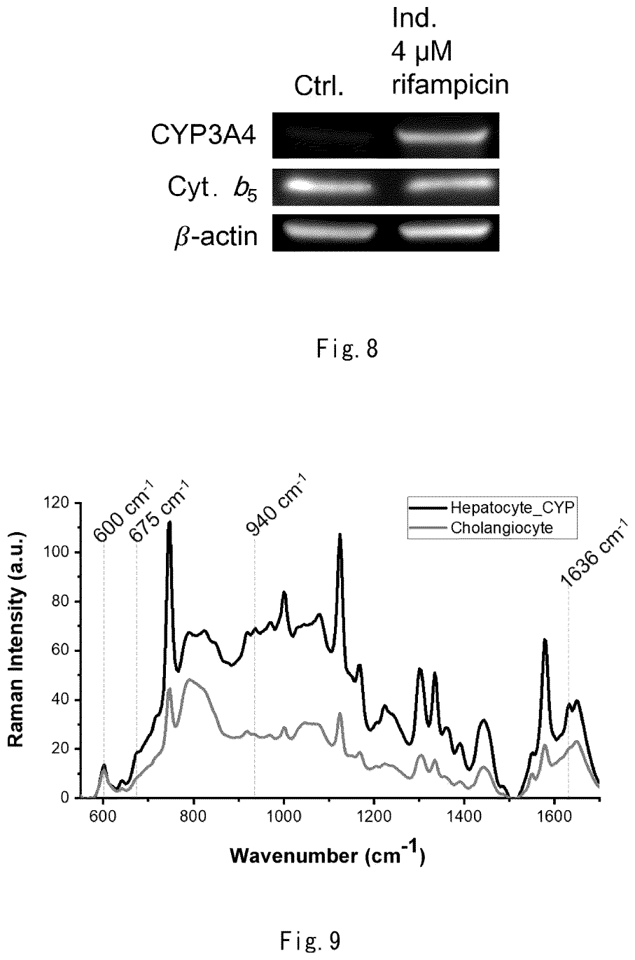

Next, the results of the luminescence method and Western blotting performed in parallel with the Raman observation are explained with reference to FIG. 7 and FIG. 8. It was confirmed by the luminescence method and Western blotting that CYP3A4, one type of the CYP enzyme group, was induced in the cell group of cultured hepatocytes.

The bar graph in FIG. 7 shows that CYP activity increased in the cell group in which CYP3A4 was induced using Rifampicin as compared with the cell group in which CYP3A4 was not induced.

In the Western blotting photograph in FIG. 8, no band is observed in the CYP3A4 non-induced cell group. On the other hand, in the cell group in which CYP3A4 was induced using Rifampicin, the expression level of CYP enzyme increased.

By Raman observation, it was confirmed that Raman signals at wavenumbers of 1370 and 1636 cm⁻¹ increased due to the induction of the CYP enzyme group using Rifampicin. From the average spectrum, the peaks and waveforms of, in addition to wavenumbers of 1370 and 1636 cm⁻¹, conventionally-known Raman signals of reduced heme (wavenumbers 600, 675, and 750 cm⁻¹) (Non Patent Literature 4), Raman signal (wavenumber 940 cm⁻¹) indicating glycogen related to glucose metabolism in liver cells (Non Patent Literature 16), and Raman scattering signal (wavenumber 1000 cm⁻¹) indicating phenylalanine, an essential amino acid abundantly contained in the cytoplasm, were identified.

### <Procuring Intracellular Distribution of Raman Signals Caused by Induction of CYP Enzyme Group in Human Hepatocytes (FIG. 9)>

Successively, identification of the biomolecules indicated by the detected Raman signals was attempted by investigating the intracellular distribution of Raman signals caused by the induction of CYP enzyme group. Using the Raman data measured according to the procedure described in the above-mentioned (5), Raman signals indicating each biomolecule were extracted according to the methods described in the above-mentioned (6) and (7), and the signal intensity distribution within the cell was determined.

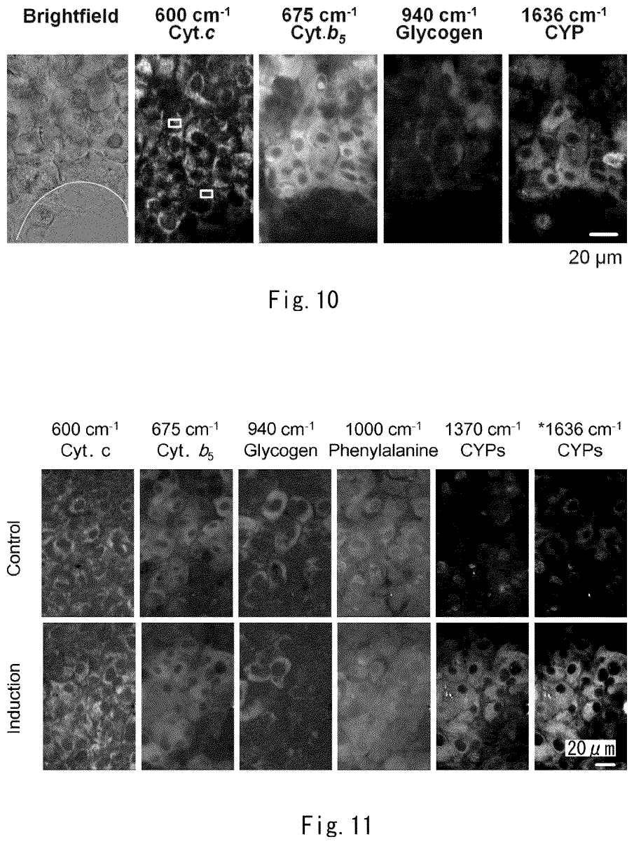

It is known that Raman signals around wavenumbers of 1370 cm⁻¹ and 1636 cm⁻¹ are derived from type b and type c of oxidized heme (Non Patent Literature 4). The CYP enzyme group induced by Rifampicin is a type b heme protein (Non Patent Literature 12). Therefore, the aforementioned Raman observation results are consistent with the prediction that the Raman signals at wavenumbers of 1370 and 1636 cm⁻¹ are caused by the CYP enzyme group having type b heme as a prosthetic group. As mentioned above, the molecules of these CYP enzyme group are mainly occupied by CYP3A4. The Raman signals with wavenumbers of 1370 and 1636 cm⁻¹ are Raman signals detected only in hepatocytes. The results are also consistent with the fact that the CYP enzyme group is expressed only in hepatocytes and is not expressed in bile duct epithelial cells. A detailed explanation is given below using FIG. 9 and FIG. 10.

FIG. 9 is a graph showing Raman spectra of hepatocytes and bile duct epithelial cells. The black line indicates the average Raman spectrum within a predetermined region of a group of hepatocytes. The gray line indicates the average Raman spectrum within a predetermined region of the bile duct epithelial cell group. It is represented by the Raman scattering intensity (Raman scattering signal intensity) on the vertical axis and the Raman shift (wavenumber) on the horizontal axis. As shown in the graph of FIG. 9, the Raman signal at a wavenumber of 1636 cm⁻¹ can be confirmed only in hepatocytes (black line), and cannot be confirmed in bile duct epithelial cells (gray line). Although not clearly shown in FIG. 9, the Raman signal with a wavenumber of 1370 cm⁻¹ also exhibits the same tendency as the Raman signal with a wavenumber of 1636 cm⁻¹.

The predetermined regions where the average Raman spectra of the hepatocyte group and the bile duct epithelial cell group were obtained are explained using FIG. 10. FIG. 10 shows Raman images constructed based on specific Raman peaks. The leftmost part of FIG. 10 is a bright field photograph. The upper side of the white line drawn on the bright field photograph indicates hepatocytes, and the lower (inner) side of the white line indicates bile duct epithelial cells. In the second photograph from the left, the predetermined region of the hepatocyte group indicated by the black line in FIG. 9 is indicated by the upper white square, and the predetermined region of the bile duct epithelial cell group indicated by the gray line in FIG. 9 is indicated by the lower white square. As mentioned above, FIG. 9 is a graph showing the average Raman spectrum within the predetermined region. FIG. 10 shows Raman images at respective wavenumbers of 600, 675, 940, and 1636 cm⁻¹. Although not clearly shown in FIG. 10, the Raman image with a wavenumber of 1370 cm⁻¹ also exhibits the same tendency as the Raman image with a wavenumber of 1636 cm⁻¹.

These facts and experimental results suggest that wavenumbers of 1370 and 1636 cm⁻¹ may be caused by the CYP enzyme group. They can also be used to distinguish between hepatocytes and bile duct epithelial cells. For such distinction, a Raman signal of 675 cm⁻¹ can be used in addition to the Raman signal of the above-mentioned wavenumber.

In order to determine whether the Raman signals at wavenumbers of 1370 and 1636 cm⁻¹ were derived from type c or type b heme, the difference in the intracellular distribution of type c heme and type b heme was focused on. It has been reported that the CYP enzyme group, which is type b heme, is mainly present in the endoplasmic reticulum (Non Patent Literature 17). On the other hand, type c heme is mainly distributed on mitochondrial membranes and is known to be involved in energy metabolism. It has been reported that when observing the inside of a cell, the distribution of type c heme matches the surface shape of mitochondria (Non Patent Literature 3). Raman images were created using the Raman signals representing the various types of reduced heme mentioned above, and the intensity distribution of Raman signal was compared between the hepatocytes in which the CYP enzyme group was induced and the non-induced cell group (FIG. 11).

As shown in FIG. 11, Glycogen (940 cm⁻¹) and the CYP enzyme group (1370, 1636 cm⁻¹) indicate biomolecules that are caused by the metabolic function of the liver. Other Raman peaks (600, 675 cn˜¹) indicate heme proteins different from the CYP enzyme group. The Raman signal at 600 cm⁻¹ indicates reduced cytochrome c, and the Raman signal at 675 cm⁻¹ indicates reduced cytochrome b. A Raman image constructed using Raman signals of each wavenumber shows the concentration distribution of molecules caused by each Raman signal. The Raman signal at 750 cm⁻¹ is derived from reduced cytochrome c and cytochrome b, and shows a distribution similar to superposed images at 600 cm⁻¹ and 675 cm⁻¹ (not shown). In cells in which CYP3A4 is induced, the concentration of glycogen molecules decreases and the concentration of CYP enzyme group molecules increases. The concentration distribution of other heme proteins does not correlate with the induction of CYP3A4.

Since cytochrome c is mainly distributed in mitochondria, Raman images of reduced cytochrome c show the distribution of mitochondria. Since cytochrome b is mainly distributed in the ER (endoplasmic reticulum), Raman images of reduced cytochrome b show the distribution of ER.

In the Raman signal of reduced heme protein and the Raman signal showing phenylalanine (1000 cm⁻¹), changes in signal intensity due to the induction of the CYP enzyme group using Rifampicin could not be confirmed. On the other hand, in the Raman signal indicating glycogen, it could be confirmed that the signal intensity reduces by inducing the CYP enzyme group by using Rifampicin, as compared with the cell group in which the CYP enzyme group was not induced.

As shown in FIG. 11, the intracellular intensity distribution of the Raman signal with a wavenumber of 600 cm⁻¹ was localized in fine structures such as mitochondria. On the other hand, the intracellular intensity distribution of the Raman signal with a wavenumber of 1636 cm⁻¹ was spread throughout the cytoplasm. The endoplasmic reticulum, in which the CYP enzyme group (type b heme protein) is distributed, originally has a finer network structure than mitochondria (Non Patent Literature 18). Therefore, with the spatial resolution (about 260 nm) of the microscope used in this experiment, it is considered that a clear image showing that the distribution of Raman signals matches the structure thereof cannot be observed.

Therefore, the intensity distribution of the Raman signals at 1370 and 1636 cm⁻¹ in FIG. 11 is consistent with the observation that endoplasmic reticulum is distributed throughout the cytoplasm.

A Raman signal with a wavenumber of 675 cm⁻¹ is obtained when cytochrome b takes a reduced form (Non Patent Literature 19). Cytochrome b is a type b heme protein similar to the CYP enzyme group, and its signal distribution indicates a cytoplasm, similar to the distribution of Raman signals at wavenumbers of 1370 and 1636 cm⁻¹. Therefore, it can be said that the Raman signals with wavenumbers of 1370 and 1636 cm⁻¹ are type b heme proteins distributed in the endoplasmic reticulum.

The intensity distribution of Raman signals with a wavenumber of 940 cm⁻¹ derived from glycogen, which is related to sugar metabolism in liver cells, was also confirmed in addition to heme proteins. The results thereof are shown in FIG. 11. It could be confirmed that the Raman signal derived from glycogen was distributed only in hepatocytes having metabolic ability, similar to the CYP enzyme group.

By detecting changes in the intensity distribution of Raman signals indicating various heme proteins and Raman signals with a wavenumber of 940 cm⁻¹, molecular information regarding the maturity level and drug response of hepatocytes can be measured.

<Comparison of Raman Observation Results and Immunostaining Results of Human Liver Cell with CYP Enzyme Group Induction (FIG. 12)>

In order to confirm that the Raman signal with wavenumbers of 1370 and 1636 cm⁻¹ is derived from the CYP enzyme group but is not derived from cytochrome b₅, the Raman observation results and immunostaining results of human liver cells in which the CYP enzyme group was induced using Rifampicin were compared. CYP3A4 and cytochrome b₅ were immunostained and fluorescence was observed. As mentioned above, CYP3A4 has the highest expression level among the CYP enzyme group induced by Rifampicin. On the other hand, cytochrome b₅ is a type b heme protein other than the CYP enzyme group that is known to exist in the endoplasmic reticulum. FIG. 12 shows fluorescence images obtained by the above-mentioned method (8).

When distribution of cytochrome b₅ and CYP shown in FIG. 12 and the distributions of the Raman signal of type b reduced heme protein (wavenumber 675 cm⁻¹) shown in FIG. 11 and the Raman signal (1370, 1636 cm⁻¹) of type b oxidized heme protein are compared, the both indicate the entire cytoplasm. It has been reported that cytochrome b₅ is also distributed in the endoplasmic reticulum like the CYP enzyme group. The Raman observation results by the inventors are also consistent with the search results previously reported for cytochrome b₅.

Therefore, it is confirmed as follows that the Raman signal with wavenumber of 1370 and 1636 cm⁻¹ is not derived from cytochrome b₅. That is, when the intensities of Raman signals are compared as shown in FIG. 11, first, Raman signals at wavenumbers of 1370 and 1636 cm⁻¹ increase in cells induced using Rifampicin. In contrast, there was no significant difference in the intensity change of the Raman signal at a wavenumber of 675 cm⁻¹ between the induced cells and non-induced cells. It is already known from the aforementioned search results that the Raman signal with a wavenumber of 675 cm⁻¹ indicates reduced cytochrome b. The immunostaining results also showed a similar tendency. The fluorescence intensity of the label for CYP3A4 increased only in cells in which the CYP enzyme group was induced. No significant difference was found in the fluorescence intensity of the label for cytochrome b₅ between the induction group and the control group. Therefore, it can be said that the Raman signals at wavenumbers of 1370 and 1636 cm⁻¹ derived from type b oxidized heme protein are mainly derived from the CYP enzyme group rather than cytochrome b₅.

In the Raman image at wavenumbers 1370 and 1636 cm⁻¹ shown in FIG. 11 and the CYP3A4 fluorescence image shown in FIG. 12, the intensity distribution of the Raman signal and the intensity distribution of fluorescence do not match in some cells. This difference in distribution is considered to derive from CYP molecule other than CYP3A4. This is because only CYP3A4 can be specifically detected in the fluorescence image, whereas all CYP molecules are detected in the Raman image. As mentioned above, Rifampicin, which was used as an inducing agent in this experiment, has been reported to induce CYP2B6, CYP2C8, CYP2C9, and CYP2C19 (Non Patent Literature 15), which are molecules included in the CYP enzyme group other than CYP3A4.

### Example 2

### <Detection of Dependence of CYP Activity on Inducing Agent Concentration >

In this Example, an attempt was made to detect the dependency of CYP activity on inducing agent concentration by Raman observation. As mentioned above, the inducing agent (Rifampicin) induces the expression of CYP3A4 in human liver cells (HepaRG cells). Therefore, CYP3A4 in human liver cells (HepaRG cells) was induced using different concentrations of the inducing agent (Rifampicin). Liver cells were seeded, cultured, and induced according to the procedures of the above-mentioned (1) and (2). The inducing agent (Rifampicin) was added at concentrations of 0, 0.04, 0.4, or 4 μM. As a control experiment for Raman observation, the activity of CYP3A4 expressed in the cells was detected using a luminescence method according to the procedure shown in the above-mentioned (3) in a part of the cells after induction. Raman observation was performed on other cells according to the procedure shown in the above-mentioned (5). Raman spectra were obtained by irradiating a rectangular region of 133.3 μm in length×84 μm in width with a laser at a scanning pitch of 1 μm at three locations in the culture dish. Noises were removed from the obtained Raman spectra according to the methods described in the above-mentioned (6) and (7).

In the above-mentioned Example, it was assumed that the Raman signals with wavenumbers of 1370 and 1636 cm⁻¹ were derived from the CYP enzyme group and correlated with the activity of the CYP enzyme group. Therefore, as shown in FIG. 13, a Raman image of the rectangular region was reconstructed using the distribution of Raman signals at a wavenumber of 1636 cm⁻¹ (oxidized type b heme protein). The Raman signal with a wavenumber of 1636 cm⁻¹ is uniformly distributed throughout the cytoplasm. Note that FIG. 13 shows a region of 84×84 μm cut out from the obtained Raman image.

Although not clearly shown, the changes in the distribution of the Raman signal at a wavenumber of 1370 cm⁻¹ and the changes in the intensity of the Raman signal also exhibited the same tendency as those of a wavenumber of 1636 cm⁻¹.

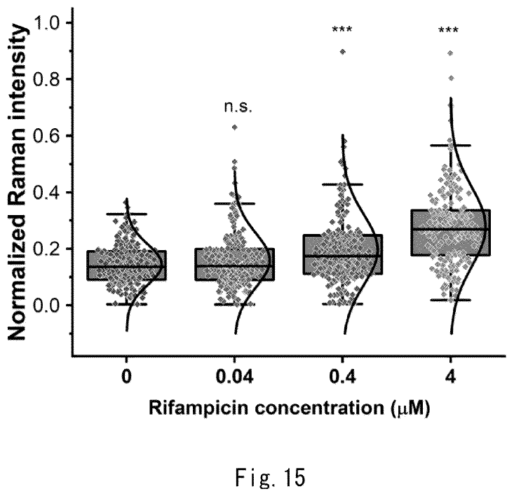

FIG. 14 and FIG. 15 show the calculation results of the average spectrum of the region of hepatocytes. In a peak including a wavenumber of 1636 cm⁻¹, the intersection of a straight line connecting both ends of the base of the peak and a straight line drawn perpendicularly from the vertex of the peak waveform to the wavenumber axis is set as the point of origin, and the height to the peak vertex was extracted as the signal intensity.

In FIG. 14, the thin gray bar graph indicates the activity of CYP3A4 detected using a luminescence method. On the other hand, the dark gray bar graph indicates the Raman signal at a wavenumber of 1636 cm⁻¹ extracted from the Raman spectrum averaged in the region of hepatocytes. The Raman signal intensity at a wavenumber of 1636 cm⁻¹ for each added concentration of Rifampicin was compared with the results of CYP3A4 activity using a luminescence method. It was confirmed that as the concentration of the administered Rifampicin increased, the Raman signal intensity at a wavenumber of 1636 cm⁻¹ indicating the CYP enzyme group and the activity of CYP3A4 increased. Therefore, it can be said that the Raman signal with a wavenumber of 1636 cm⁻¹ has a positive correlation with the activity of CYP3A4.

FIG. 15 is a box-whisker showing the dispersion in CYP enzyme group activity for each cell. Each plot is a Raman signal with a wavenumber of 1636 cm⁻¹ extracted from the average spectrum of each cell according to the method described above. It can be confirmed that even under conditions where the concentration of the administered Rifampicin is constant, the induced activities of CYP enzyme group vary from cell to cell. The detection of the activity of CYP enzyme group using Raman signals indicates that not all cells in the culture dish increase the CYP activity uniformly. Furthermore, it was confirmed that the median value of the plot for each dosage concentration condition of Rifampicin increased along with the dosage concentrations of Rifampicin. This increase in median value was statistically significant. It also corresponds well with the increase in CYP3A4 activity.

### Example 3

<Detection of CYP Activity in Liver Cells with Various Inducing Agents Added>

In this example, an attempt was made to observe differences in the response of the activity of the CYP enzyme group depending on the kind of inducing agent. Four kinds of inducing agents (Rifampicin, Phenytoin, Dexamethasone, Carbamazepine) known to induce CYP expression in human liver cells (HepaRG cells) were used. CYPs in human liver cells (HepaRG cells) were induced using these, and detection of the activity of the CYP enzyme group by Raman observation was attempted. These inducing agents activate the transcription factor PREGNANT X RECEPTOR:PXR. Activated PXR migrates from the cytoplasm to the nucleus, forms a hetero dimer with the RETINOID X RECEPTOR (RXR), and binds to the promoter sequence. Transcription of the target gene proceeds (Non Patent Literatures 20 to 22), resulting in enhanced expression of CYP enzyme group. Transcription of CYP3A4 is mainly promoted through PXR.

According to the procedures of the above-mentioned (1) and (2), seeding, culture, and induction of human liver cells (HepaRG cells) were performed. Four kinds of inducing agents (Rifampicin, Phenytoin, Dexamethasone, Carbamazepine) were added at concentrations of 4, 100, 100, or 100 μM, respectively.

In addition, as a control, non-induced liver cells cultured in a medium containing no inducing agent (MIL600, ADD650, BIOPREDIC International) were also prepared. In order to perform luminescence method and Raman observation, cells were separated from these cell groups for each observation. A part of the human liver cells (HepaRG cells) after induction was used for the luminescence method as a control experiment for Raman observation, and the remaining human liver cells (HepaRG cells) were used for Raman observation. The luminescence method and Raman observation were performed according to the procedures described in the above-mentioned (3) and (5), respectively. From the above, the activity of CYP3A4 in human liver cells (HepaRG cells) was measured. As described above, the Raman signal at 1636 cm⁻¹ is expected to correlate well with CYP activity. In the analysis of the Raman observation results, in the above-mentioned (6) and (7), the Raman signal at 1636 cm⁻¹ was extracted and compared with the detection results of CYP3A4 using the luminescence method.

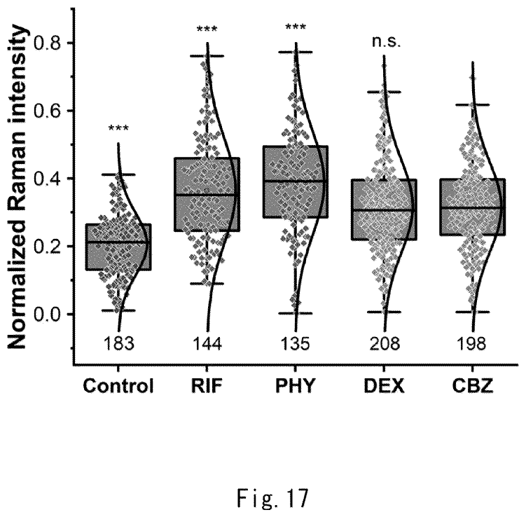

FIG. 16 and FIG. 17 show the measurement results of the activity of CYP enzyme group when induced using Rifampicin, Phenytoin, Carbamazepine, and Dexamethasone as inducing agents. The activity of CYP3A4 induced by various agents could be measured using Raman signals at a wavenumber of 1636 cm⁻¹. FIG. 16 shows CYP3A4 activity (thin gray) and Raman signal (dark gray) detected using a luminescence method. The black bar graph in FIG. 16 is a plot of Raman signal intensity at a wavenumber of 1636 cm⁻¹, which is the Raman peak of the CYP enzyme group, after averaging the Raman spectra detected from the region of hepatocytes, and the thin gray bar graph indicates CYP activity detected by a luminescence method. For extraction of signals in this case, in a peak including a wavenumber of 1636 cm⁻¹, the intersection of a straight line connecting both ends of the base of the peak and a straight line drawn perpendicularly from the vertex of the peak waveform to the wavenumber axis is set as the point of origin, and the height to the peak vertex was obtained as the signal intensity.

FIG. 17 is a box-whisker showing the dispersion in CYP enzyme group activity for each cell. Each plot is a Raman signal with a wavenumber of 1636 cm⁻¹ extracted from the average spectrum of each cell in the same manner as above. Similar to the results in FIG. 15, the induced activities of CYP enzyme group vary from cell to cell even under certain constant culture conditions. In addition, not all cells in the culture dish increase the CYP activity uniformly. Furthermore, the increase and decrease in the median values of the plots under each culture condition are consistent with the variation in CYP3A4 activity detected by the luminescent method. The increase in the median value of plot was statistically significant.

Although not clearly shown, the changes in the distribution of the Raman signal at a wavenumber of 1370 cm⁻¹ and the changes in the intensity of the Raman signal also exhibited the same tendency as those of a wavenumber of 1636 cm⁻¹.

### Example 4

### <Detection of Suppression of CYP Activity by IL-6 (Interleukin-6)>

In this Example, an attempt was made to observe suppression of CYP activity. Interleukin-6 (IL-6), which is known to suppress the activity of the CYP enzyme group in human liver cells (HepaRG cells), was used to suppress CYP activity. After suppressing or down-regulating the activity of the CYP enzyme group in human liver cells (HepaRG cells), detection of the activity of the CYP enzyme group was attempted by Raman observation. IL6 is a primary mediator substance of the acute phase response and is one of the cytokines that play a central role in multiple chronic inflammatory diseases. The release of IL-6 occurs when cells are exposed to inflammatory or infectious stress. IL-6 is known to suppress the expression of CYP3A4 mRNA in HepaRG cells, a liver cancer cell line (Non Patent Literature 23). As a result of suppression of mRNA expression, expression of CYP3A4 protein is suppressed.

For down-regulation of the activity of CYP enzyme group by using IL-6, liver cells that underwent seeding and cultured according to the procedure the above-mentioned (1) were used. Prior to administration of IL-6, induction of all types of CYP enzyme, i.e., induction of non-specific to CYP enzyme type, was performed. Non-specific induction of CYP enzyme group was performed by culturing HepaRG cells for 2 days in a medium containing DMSO at a concentration of 2% (MIL600, ADD620, BIOPREDIC International). Thereafter, the cells were cultured for 48 hr in a medium (MIL600, ADD650, BIOPREDIC International) containing IL-6 (206-IL-010/CF, R&D systems) at a concentration of 2 ng/ml. In addition, as a control, liver cells cultured for 48 hr in a medium not containing IL-6 (MIL600, ADD650, BIOPREDIC International) were also prepared. For use in luminescence method and Raman observation, cells were separated from these cell groups for each observation.

A part of the human liver cells (HepaRG cells) after induction and administered of IL-6 was used for the luminescence method as a control experiment for Raman observation, and the remaining human liver cells (HepaRG cells) were used for Raman observation. The luminescence method and Raman observation were performed according to the procedures described in the above-mentioned (3) and (5), respectively, and the activity of CYP3A4 in human liver cells (HepaRG cells) was measured. In the analysis of the Raman observation results, in the above-mentioned (6) and (7), the Raman signal at 1636 cm⁻¹ expected to correlate well with CYP activity was extracted and compared with the results of CYP3A4 using the luminescence method.

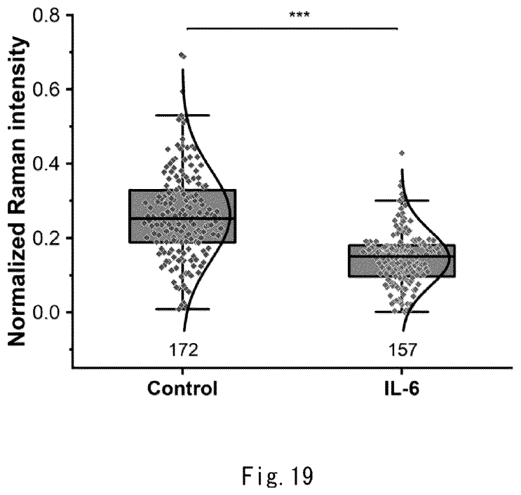

FIG. 18 and FIG. 19 show the results detected using Raman signals as to the downregulation of CYP activity caused by administration of IL-6. As a result, it was detected by a luminescence test that the activity of CYP3A4 also decreases, which is in good agreement with the Raman measurement results. The bar graph of FIG. 18 shows CYP3A4 activity (thin gray) and Raman signal (dark gray) detected using a luminescence method. The dark gray bar graph in FIG. 18 shows Raman signal intensity plotting Raman signal at a wavenumber of 1636 cm⁻¹, which is the Raman peak of the CYP enzyme group, after averaging the Raman spectra from the region of hepatocytes, and the thin gray bar graph indicates CYP activity detected by a luminescence method. For extraction of signals, in a peak including a wavenumber of 1636 cm⁻¹, the intersection of a straight line connecting both ends of the base of the peak and a straight line drawn perpendicularly from the vertex of the peak waveform to the wavenumber axis is set as the point of origin, and the height to the peak vertex was obtained as the signal intensity.

FIG. 19 is a box-whisker showing the dispersion in CYP enzyme group activity for each cell. Each plot is a Raman signal with a wavenumber of 1636 cm⁻¹ extracted from the average spectrum of each cell in the same manner as above. Similar to the results in FIG. 15, the activities of CYP enzyme group vary from cell to cell even under certain constant culture conditions. In addition, not all cells in the culture dish decrease in the CYP activity uniformly. Furthermore, the decrease in the median values of the plots under each culture condition (presence or absence of administration of CYP enzyme group expression inhibitor) is consistent with the variation in CYP3A4 activity detected by the luminescent method. The decrease in the median value of the observed plot was statistically significant.

Although not clearly shown, the changes in the distribution of the Raman signal at a wavenumber of 1370 cm⁻¹ and the changes in the intensity of the Raman signal also exhibited the same tendency as those of a wavenumber of 1636 cm⁻¹.

### Example 5

<Time-Course Raman Observation of Changes in CYP Activity Associated with Liver Cell Seeding>

In this Example, an attempt was made to detect, by Raman observation, changes over time in the activity of CYP enzymes depending on the number of culture days of human liver cells (HepaRG cells). First, frozen human liver cells (HepaRG cells) (HPR116, BIOPREDIC International) were thawed. It is known that HepaRG cells exhibit high CYP enzyme group activity after seeding, and the activity decreases over the next 48 hr.

According to the procedures of the above-mentioned (1), seeding and culture of human liver cells (HepaRG cells) were performed. In order to perform luminescence method and Raman observation, cells were separated from these cell groups for each observation. The activity of CYP enzyme group was detected using luminescence method and Raman observation 6 hr, 24 hr, and 48 hr after seeding HepaRG cells.

A part of the prepared human hepatocytes (HepaRG cells) was used for the luminescence method as a control experiment, and the remaining human hepatocytes (HepaRG cells) were used for Raman observation. The luminescence method and Raman observation were performed according to the procedures described in the above-mentioned (3) and (5), respectively, and the activity of CYP3A4 in human hepatocytes (HepaRG cells) was measured. In the Raman observation of this Example, different cell groups that were seeded and cultured at the same timing were observed at each time point. In the analysis of the Raman observation results, in the above-mentioned (6) and (7), the Raman signal at 1636 cm⁻¹ expected to correlate well with CYP activity was extracted and compared with the results of CYP3A4 using the luminescence method.

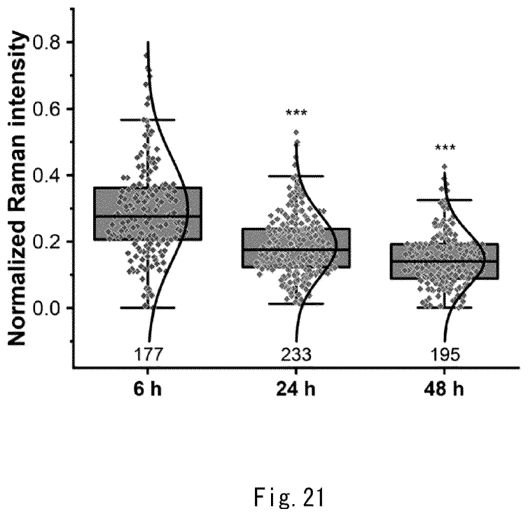

FIG. 20 and FIG. 21 show the detection results of changes over time in the CYP activity of HepaRG cells by using a luminescence method and Raman observation, respectively. The time course changes in the activities of CYP enzyme group after seeding HepaRG cells could be measured using Raman signal at a wavenumber of 1636 cm⁻¹. The dark gray bar graph of FIG. 20 shows a plot of Raman signal intensity at a wavenumber of 1636 cm⁻¹, which is the Raman peak of the CYP enzyme group, after averaging the Raman spectra from the region of hepatocytes, and the thin gray bar graph indicates CYP activity detected by the luminescence method. For extraction of signals, in a peak including a wavenumber of 1636 cm⁻¹, the intersection of a straight line connecting both ends of the base of the peak and a straight line drawn perpendicularly from the vertex of the peak waveform to the wavenumber axis is set as the point of origin, and the height to the peak vertex was obtained as the signal intensity.

FIG. 21 is a box-whisker showing the dispersion in CYP enzyme group activity for each cell. Each plot is a Raman signal with a wavenumber of 1636 cm⁻¹ extracted from the average spectrum of each cell in the same manner as above. Similar to the results in FIG. 15, the activities of CYP enzyme group vary from cell to cell even under certain constant culture conditions. In addition, not all cells in the culture dish lose the CYP activity uniformly. Furthermore, the decrease in the median values of the plots under each culture condition (difference in time point) is consistent with the variation in CYP3A4 activity detected by the luminescent method. The decrease in the median value of the plot was statistically significant.

From the results of Examples 2 to 5, a positive correlation was confirmed between the Raman signal at a wavenumber of 1636 cm⁻¹ indicating the oxidized CYP enzyme group and the activity of CYP3A4 detected by the luminescence method. From this, it can be said that the activity of the CYP enzyme group can be measured using a Raman signal with a wavenumber of 1636 cm⁻¹.

Although not clearly shown, the changes in the distribution of the Raman signal at a wavenumber of 1370 cm⁻¹ and the changes in the intensity of the Raman signal also exhibit the same tendency as those of a wavenumber of 1636 cm⁻¹. Therefore, Raman signals with a wavenumber of 1370 cm⁻¹ can also be used to measure CYP enzyme activity.

### Example 6

### <Inhibition of CYP Activity by Azamulin>

In this Example, an attempt was made to detect, by Raman observation, inhibition of the activity of CYP enzymes of human liver cells (HepaRG cells). Azamulin is known as an inhibitor that specifically acts on CYP3A (including CYP3A4) as a mechanism-based inhibitor (MBI) (Non Patent Literature 24). Therefore, Azamulin inhibits CYP3As activity in human liver cells (HepaRG cells).

Azamulin metabolized by the catalytic action of CYP3As produces highly reactive metabolites. The metabolites irreversibly form covalent bonds with CYP3As including CYP3A4, thereby inhibiting the catalytic activity (metabolic ability) of CYP3As. When CYP3As are inhibited, drugs in the body are not metabolized, resulting in increased drug concentrations in the blood. As a result, the risk of changes in therapeutic efficacy and the occurrence of serious side effects increases. Therefore, it is important in the evaluation technique for CYP3As activity to detect a decrease in CYP3As activity (drug metabolic ability) caused by an inhibitor.

Liver cells were seeded, cultured, induced, and inhibited according to the procedures of the above-mentioned (1) and (2). An inducing agent (Rifampicin) and an inhibitor (Azamulin) were added at concentrations of 4 μM and 10 μM, respectively. As a control, human liver cells (HepaRG cells) cultured in a medium not containing an inhibitor (Azamulin) (MIL600, ADD650, BIOPREDIC International) were also prepared. In order to perform luminescence method and Raman observation, cells were separated from these cell groups for each observation. A part of the prepared human liver cells (HepaRG cells) was used for the luminescence method as a control experiment for Raman observation, and the remaining human liver cells (HepaRG cells) were used for Raman observation. The luminescence method and Raman observation were performed according to the procedures described in the above-mentioned (3) and (5), respectively, and the activity of CYP3A4 in human liver cells (HepaRG cells) was measured. In the analysis of the Raman observation results, in the above-mentioned (6) and (7), the Raman signal at 1636 cm⁻¹ expected to correlate well with CYP activity was extracted and compared with the results of CYP3A4 using the luminescence method.

FIG. 22 and FIG. 23 show the activities of CYP enzyme group detected by the luminescence method and Raman observation, respectively. In FIG. 22, CYP3A4 activity is measured by the luminescence method depending on the presence or absence of CYP3A4 inhibition by Azamulin. As mentioned above, CYP is induced with Rifampicin before administration of Azamulin.

From the detection results by the luminescent method shown in FIG. 22, it was confirmed that 82% of all CYP3A4 lost activity by inhibiting the CYP enzyme group for 5 min with Azamulin.

FIG. 23 shows the relationship between the presence or absence of CYP3A4 inhibition by Azamulin and the Raman signal at a wavenumber of 1636 cm⁻¹. For extraction of Raman scattering signals, in a peak including a wavenumber of 1636 cm⁻¹, the intersection of a straight line connecting both ends of the base of the peak and a straight line drawn perpendicularly from the vertex of the peak waveform to the wavenumber axis is set as the point of origin, and the height to the peak vertex was obtained as the signal intensity. Before administration of Azamulin, CYP was induced with Rifampicin. FIG. 23 shows that administration of Azamulin reduces the Raman signal at a wavenumber of 1636 cm⁻¹. Each plot is a Raman signal with a wavenumber of 1636 cm⁻¹ extracted from the average Raman spectrum of each cell. The Raman signal with a wavenumber of 1636 cm⁻¹ varies among individual cells.

This indicates differences in the activity of the CYP enzyme group among cells. The median value of the plot decreased in human liver cells (HepaRG cells) administered with Azamulin, as compared with the cell group induced with Rifampicin but not administered with Azamulin. From this, it can be said that inhibition of the activity of CYP enzyme group could be detected by observing the Raman signal at a wavenumber of 1636 cm⁻¹. It has also been confirmed that the amount of decrease in the median value of the plot is statistically significant.

Based on the prior art and the experimental results to date, it is understood that the signal of oxidized heme b (wavenumber 1636 cm⁻¹) is correlated with the CYP enzyme activity of the CYP enzyme group in cells. More specifically, by considering the following (1) to (7), it is understood that the enzyme activity of the CYP enzyme group has a clear correlation with the number of molecules of the oxidized CYP enzyme group:
(1) The main premise is that in humans, CYP3A4 accounts for the majority (about 30%) of the CYP present in the liver. (Pharmacology & Therapeutics 138 (2013) 103-141. Cytochrome P450 enzymes in drug metabolism: Regulation of gene expression, enzyme activities, and impact of genetic variation)
(2) When Rifampicin is used as a CYP enzyme inducing agent, the signal at 1636 cm⁻¹ increased in a dose-dependent manner with the addition of Rifampicin and the evaluation results by an existing CYP3A4 enzyme activity evaluation method show high correlation (FIGS. 13, 14, 15).
(3) Even when a CYP inducing agent other than Rifampicin is used, an increase in the signal at 1636 cm and the evaluation results by the existing CYP3A4 enzyme activity evaluation method show high correlation (FIGS. 16, 17).
(4) The evaluation results by the existing CYP enzyme activity evaluation method and the evaluation results by the signal at 1636 cm also show high correlation with respect to the CYP activity suppressing tendency by IL-6 (FIGS. 18, 19).
(5) Even when induction of CYP enzyme group is not performed, the evaluation results of the CYP enzyme activity by the existing evaluation method and the evaluation results by signal at 1636 cm⁻¹ show high correlation in time course changes in the decrease of CYP activity (FIGS. 20, 21).
(6) Even in an experiment system with the addition of Azamulin, a competition inhibitor of CYP3A4, the evaluation results of the CYP enzyme activity by the existing evaluation method and the evaluation results by signal at 1636 cm⁻¹ show high correlation in the decrease of CYP activity (FIGS. 22, 23). Considering the above (1) to (6), the enzyme activity of the CYP enzyme group has a very strong correlation with the amount of molecules of the oxidized CYP enzyme group, and the enzyme activity of CYP enzyme group can be measured by measuring the amount of molecules of the oxidized CYP enzyme group.
   Furthermore, the correlation between the amount of molecules of the oxidized CYP and the enzyme activity of the CYP enzyme group can also be explained from the theoretical interpretation shown in (7).
(7) In the reaction cycle of the CYP enzyme group, the enzyme reaction cycle rotates by repeating the structural change of “oxidized CYP→reduced CYP→oxidized CYP→reduced CYP”. Addition of a competitive inhibitor does not change the amount of molecules of the CYP enzyme group, but it is considered that the reaction cycle stops at the stage of reduced CYP, resulting in a decrease in the amount of oxidized CYP molecules.

Raman observation of human liver cells (HepaRG cells) with CYP enzyme group inhibited using Azamulin was performed. The decrease in the Raman signal with a wavenumber of 1636 cm⁻¹ could be confirmed. The Raman signal with a wavenumber of 1636 cm⁻¹ is mainly caused by heme containing trivalent iron (Fe) in oxidized heme protein. Therefore, the decrease in the Raman signal with a wavenumber of 1636 cm⁻¹ confirmed in FIG. 23 is considered to be caused by the following three reasons. 1) The number of molecules of heme proteins containing heme decreases. 2) Heme proteins change from an oxidized state to a reduced state. 3) The molecular structure changes, and the molecular structure exhibiting a Raman signal with a wavenumber of 1636 cm⁻¹ decreases or disappears.

In this Example, the possibility of cause 1) is considered to be low. This is because inhibition of the activity of CYP enzyme group for 5 min using Azmulin is considered to be insufficient to change the number of heme proteins. Therefore, it is considered that causes 2) and 3) affect the Raman observation results in FIG. 23. In order to confirm the influence of cause 2), the intensity distribution of the Raman signal at a wavenumber of 675 cm⁻¹, which indicates type b reduced heme protein, and the intensity distribution of the Raman signal at wavenumber 1636 cm⁻¹. (Raman image), which indicates type b oxidized heme protein were compared. Furthermore, in order to confirm the intracellular distribution of heme protein molecules, immunostaining of the CYP enzyme group and cytochrome b₅, which are type b heme proteins contained in HepaRG cells, was performed. The results thereof are shown in FIG. 24. FIG. 24 includes a Raman image obtained by the method of the above-mentioned (5) and a fluorescence image obtained by the method of the above-mentioned (8). If the administration of Azamulin changes the oxidized CYP enzyme group into a reduced state as shown in cause 2), the intracellular Raman signal at a wavenumber of 675 cm⁻¹ should increase. However, as confirmed from the Raman observation results in FIG. 24, the Raman signal with a wavenumber of 675 cm⁻¹ did not increase. From the above results, it is considered that the remaining cause 3) is the true cause of the decrease in the Raman signal with a wavenumber of 1636 cm⁻¹. That is, it is clear that administration of Azamulin changes the molecular structure of CYP3A4, and the molecular structure exhibiting a Raman signal at a wavenumber of 1636 cm⁻¹, which reflects the enzyme activity, decreases.

Although not clearly shown, the changes in the distribution of the Raman signal at a wavenumber of 1370 cm⁻¹ and the changes in the intensity of the Raman signal also exhibited the same tendency as those of a wavenumber of 1636 cm⁻¹.

### Example 7

### <Multilateral Measurement of Liver Function Using Raman Scattering Signal Group>

In this Example, a Raman scattering signal group was used in which other Raman signals were added to the Raman signal with a wavenumber of 1370-1636 cm⁻¹ indicating CYP metabolic activity. The Raman signal with a wavenumber of 940 cmu¹ is derived from glycogen related to sugar metabolism. The wavenumber of 600 cm⁻¹ is derived from cytochrome c, which is involved in energy production. The Raman signal with a wavenumber of 2850 cm⁻¹ is related to lipid metabolism in the liver. Multilateral measurement of the cellular functions of human liver cells (HepaRG cells) was attempted by detecting these Raman scattering signal group together.

Liver cells were seeded, cultured, and induced according to the methods described in the above-mentioned (1) and (2). However, the timing of medium exchange, CYP induction, and CYP measurement was different from the above-mentioned (1) and (2). In this Example, the medium was replaced with a medium (MIL600, ADD670, BIOPREDIC International) day 1 and day 4 days after seeding, and the induction of CYP was performed on day 6 and day 7 after seeding using a medium (MIL600, ADD650, BIOPREDIC International) containing 4 μM Rifampicin. Control cells were cultured using a medium (MIL600, ADD650, BIOPREDIC International) not containing an inhibitor (Azamulin). A part of the prepared human liver cells (HepaRG cells) was used for the luminescence method as a control experiment for Raman observation, and the remaining human liver cells (HepaRG cells) were used for Raman observation. On day 1 and day 8 after seeding, the luminescence method and Raman observation were performed according to the procedures described in the above-mentioned (3) and (5), respectively, and the activity of CYP3A4 in human liver cells (HepaRG cells) was measured.

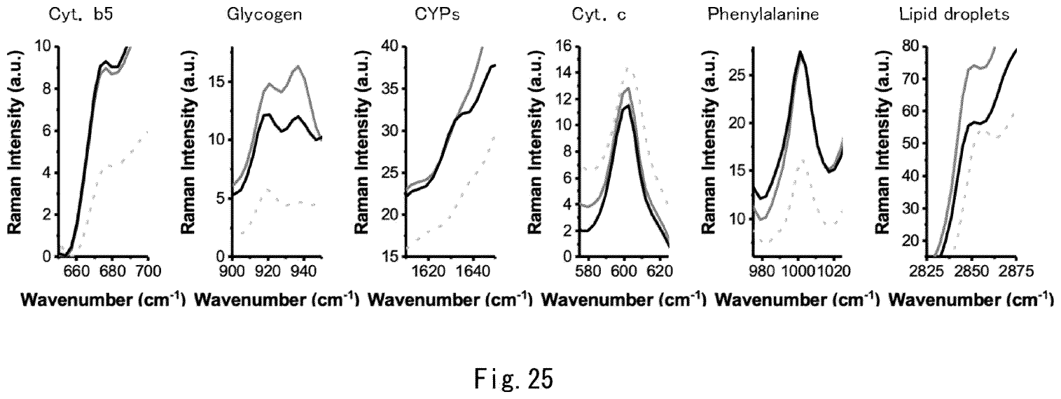

FIG. 25 shows the relationship between hepatocytes and various Raman scattering signals. In FIG. 25, the thin gray broken line shows the Raman spectrum of HepaRG cells measured on Day 1, the thin gray solid line shows the Raman spectrum measured on Day 8, and the dark gray solid line shows cells in which the CYP enzyme group was induced by 4 μM Rifampicin from Day 6 and Raman spectrum measured on Day 8. As shown in FIG. 25, it can be confirmed that the Raman signal at a wavenumber of 1636 cm⁻¹ derived from the CYP enzyme group increases according to the number of days of culture of hepatocytes or by the induction of the CYP enzyme group. The Raman signal at a wavenumber of 940 cm⁻¹ derived from glycogen increases according to the number of days of culture of hepatocytes. However, the Raman signal decreases by the administration of Rifampicin. Rifampicin has the action of promoting the decomposition of glycogen, and it was shown by this Example that this phenomenon can be observed without labeling by using a Raman microscope. The Raman signal intensity per unit cell at wavenumbers 675 and 600 cm⁻¹, which indicate cytochrome b₅ and cytochrome C, did not change. The Raman signal at a wavenumber of 2850 cm⁻¹, which indicates lipids, decreased slightly due to the induction of CYP.

Intracellular distribution of each of the above-mentioned Raman signals is shown in FIG. 26. In FIG. 26, using the Raman data measured according to the procedure described in the above-mentioned (5), Raman signals indicating each biomolecule were extracted according to the methods described in the above-mentioned (6) and (7), and the signal intensity distribution within the cell was determined. For extraction of Raman scattering signals, in a peak including a wavenumber of 1636 cm⁻¹, the intersection of a straight line connecting both ends of the base of the peak and a straight line drawn perpendicularly from the vertex of the peak waveform to the wavenumber axis is set as the point of origin, and the height to the peak vertex was obtained as the signal intensity. Raman signals at wavenumbers of 675, 940, and 1636 cm⁻¹ derived from cytochrome b₅, glycogen, and the CYP enzyme group are mainly expressed in hepatocytes and are hardly observed in bile duct epithelial cells. Raman signals at wavenumbers of 600, 1000, and 2850 cm⁻¹, which indicate cytochrome c related to energy production, the essential amino acid phenylalanine, and lipids, can be confirmed in all cells in the field of view. The distribution of each signal intensity matches with the mitochondria where cytochrome c is present, the entire cytoplasm where phenylalanine is present, and the range of existence of lipids.

Using this method, Raman signals of biomolecules related to metabolic ability from living cells could be detected under a microscope. Raman signals from biomolecules that indicate the state and structure of cells could also be simultaneously detected. The intracellular distribution of each detected biomolecule can be understood with spatial resolution at the organelle level. Therefore, when examining cellular drug responses in metabolism and energy production, comprehensive examination can be performed together with the behavior of biomolecules localized in other organelles. It is considered that the observation of Raman signals at 1636 cm⁻¹ and 940 cm⁻¹ will contribute not only to the measurement of the activities of the CYP enzyme group and sugar metabolism, but also to the elucidation of the mechanisms of those cellular functions.

Although not clearly shown, the changes in the distribution of the Raman signal at a wavenumber of 1370 cm⁻¹ and the changes in the intensity of the Raman signal also exhibited the same tendency as those of a wavenumber of 1636 cm⁻¹.

### Example 8

### <Visualization of CYP Activity in Primary Human-Derived Hepatocyte>

Frozen primary human-derived hepatocyte (PHH, Lot No. HC2-50), OptiThaw Hepatocyte isolation kit (K8000), OptiCulture media kit (K8300 M), and OptiPlate hepatocyte media (K8200) were purchased from Sekisui XenoTech, LLC. PHH was thawed using an OptiThaw hepatocyte isolation kit. To perform Raman observation, culture was performed on a 35 mm dish with a quartz bottom (SF-S-D12; Fine Plus International). Specifically, quartz on the bottom of a 35 mm dish was pre-coated with collagen, a silicon ring with an inner diameter of 15 mm was placed on the quartz, and 3.2×10⁴ cells were seeded inside the ring. After PHH adhered to the culture surface (7 hr after seeding), Raman measurement was performed. HepaRG (human hepatocyte-derived cell line) at the same density was used as a control.

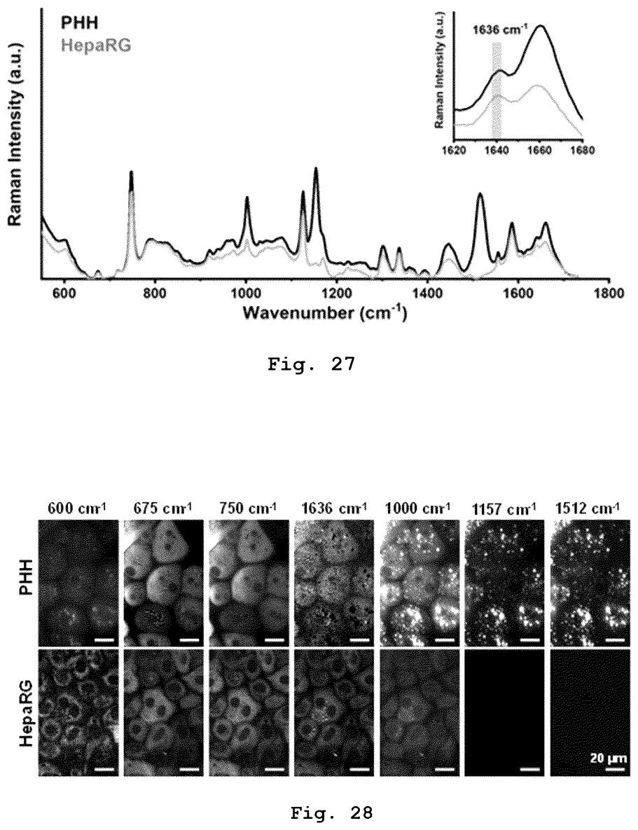

FIG. 27 shows Raman spectra of PHH and HepaRG cells. For all cells, there was a 1636 cm⁻¹ signal indicating CYP activity. FIG. 28 is a cell image of PHH and HepaRG in which Raman signals corresponding to each spectrum are visualized. It was found that intracellular CYP activity was observed in both PHH and HepaRG cells at 1636 cm⁻¹ indicating CYP activity. These results demonstrate that it is possible to detect CYP activity not only in HepaRG but also in primary liver cells and visualize intracellular distribution thereof.

### Example 9

<Visualization of CYP Activity in Hepatocyte-Like Cell (HLC) Differentiated from Human Induced Pluripotent Stem Cell (hiPSC)>

hiPSCs were seeded in a 35 mm dish with a quartz bottom pretreated with Matrigel (manufactured by BD science). The 4×10⁵ cell culture region was restricted by a silicon ring with an inner diameter of 15 mm. Differentiation was induced stepwise for 25 days in the order of definitive endoderm cells, hepatoblast-like cells, and hepatocyte-like cells (HLC) without intermediate passage. hiPS cells were cultured for 4 days in RPMI1640 medium (Sigma-Aldrich) containing 100 ng/ml Activin A (R&D Systems), 1×Gluta-MAX (Thermo Fisher Scientific), and 1×B27 Supplement Minus Vitamin A (Thermo Fisher Scientific) to induce differentiation into definitive endoderm cells. In order to induce differentiation of hepatoblast-like cells, the medium was replaced with RPMI1640 medium containing 20 ng/ml BMP4 (R&D Systems), 20 ng/ml FGF4 (R&D Systems), 1×GlutaMAX, and 1×B27 Supplement Minus Vitamin A and cultured for 5 days. Successively, cells that differentiated into hepatoblast-like cells were cultured for 5 days in RPMI1640 medium supplemented with 20 ng/ml HGF, 1×GlutaMAX, and 1×B27 Supplement Minus Vitamin A. Thereafter, induction into HLC was completed by culturing for 11 days in Hepatocyte Culture Medium Bullet Kit™ (HCM, Lonza) supplemented with 20 ng/ml oncostatin M (Osm). Thereafter, HLC cells were measured using a Raman microscope.

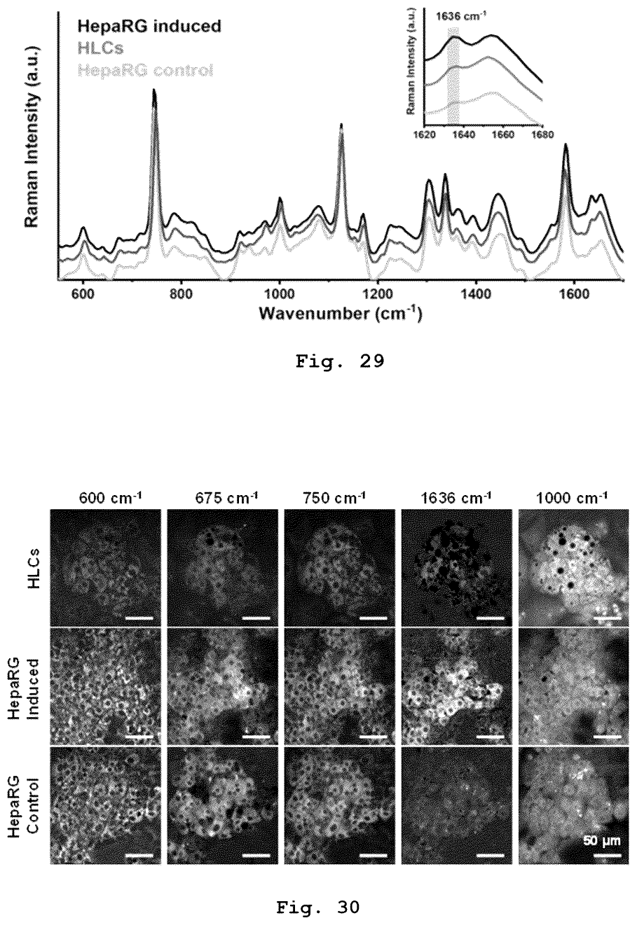

FIG. 29 shows Raman spectra of HepaRG cells in which CYP was induced with Rifampicin, HepaRG cells free of an inducing agent, and HLCs. For all cells, there was a 1636 cm⁻¹ signal indicating CYP activity. FIG. 30 shows each cell image in which Raman signals corresponding to each spectrum are visualized. It was found that intracellular CYP activity was observed at 1636 cm⁻¹ indicating CYP activity. These results demonstrate that it is possible to visualize intracellular distribution of CYPs and metabolites of not only HepaRG but also various hepatocytes.

### Application Example

Various applications according to the above-mentioned embodiments are shown below while explaining the advantages of the method for detecting the enzyme activity of the CYP enzyme group in cells. Since CYP activity is an indicator of metabolic ability, which is one of liver functions, the present method enables rapid and precise (cell organelle level) diagnosis of metabolic ability, which in turn can improve the efficiency of diagnosis in medical settings and evaluation of drug response in drug discovery. In the development of regenerative medicine technology, moreover, it can be applied to the evaluation of metabolic capacity of iPS-derived liver cells and constructed three-dimensional tissues.

Many of the prior art methods for detecting CYP activity are “destructive tests” and “invasive tests” that destroy cell tissue by one-time use of mass spectrometry method or chromogenic probe. On the other hand, in this embodiment, CYP activity can be detected with resolution at the organelle level and in a minimally invasive and label-free manner. By using the present technique, CYP activity and its distribution in three-dimensional hepatic tissue can be visualized as they are at high speed without destroying the tissue.

The inventors found that there is a correlation between the redox state of CYP metabolic enzyme molecules and CYP activity. In the above-mentioned embodiment, the activity of the CYP metabolic enzyme group is measured by detecting changes in spontaneous Raman scattered light due to the state of the molecule. The inventors found that the Raman signal derived from oxidized heme b including 1370 cm⁻¹ and 1636 cm⁻¹ is correlated with CYP activity. In the above-mentioned embodiment, the measurement of CYP activity in liver cells is performed using Raman signals. Furthermore, in the application of the above-mentioned embodiment, the activity of purified CYP protein or CYP expressed in other organ tissues is measured. In the above-mentioned embodiment, moreover, Raman signals of factors related to the metabolic function of other cells or tissues, such as reduced/oxidized heme b, reduced/oxidized heme c, and glycogen, are detected simultaneously with the measurement of the CYP activity, whereby the type of cell or tissue, degree of differentiation and the level of maturity are further evaluated.

CYP metabolic enzymes are heme proteins expressed in hepatocytes and small intestinal epithelial cells and localized in the endoplasmic reticulum (ER), and are known as a group of enzymes responsible for detoxification and drug metabolism.

The activity of CYP metabolic enzymes plays a major role in drug metabolism and is therefore used as an indicator for the measurement of liver metabolic activity in drug discovery and development. Thus, a means for detecting the activity of CYP metabolic enzymes without destroying cell tissues is strongly desired in the fields of drug discovery and regenerative medicine, and drug discovery. The inventors succeeded in non-destructively detecting the activity of CYP metabolic enzyme group by utilizing Raman scattering signals. The inventors further discovered a Raman signal that correlates not only to the amount of enzyme but also enzyme activity.

In conventional technique, Raman signals that correlate with the redox state and amount of CYP metabolic enzymes in vitro have been known, but there have been no reports of measurements in cells or tissues. In contrast, in the above-mentioned embodiment, the activity of the CYP enzyme is detected non-destructively and without staining within cells or tissues, in addition to the amount of CYP enzyme. Therefore, high-speed measurement of CYP activity, improvement of quantitativeness, and measurement of time-course changes in CYP activity in the same sample can be realized. In the above-mentioned embodiment, moreover, Raman signals derived from other indicators related to metabolism are simultaneously detected by utilizing the Raman signal. Therefore, the distribution of biomolecules caused by the metabolic state of cells or tissues can be obtained. Consequently, the above-mentioned embodiment can also be applied to the evaluation of the degree of differentiation and maturity of cells or tissues. In particular, in liver cell tissue, it is possible to evaluate the metabolism related to drug efficacy and toxicity, and the region specificity of metabolic function.

In the field of regenerative medicine, it is known that the quality of human ES/iPS cells, in particular, varies greatly depending on the number of passages and culture methods. Therefore, standardization of cell quality control techniques is demanded. Similarly, cell quality needs to be controlled also in the process of producing liver cells from ES/iPS cells. In one embodiment, quality control is performed by evaluating the metabolic ability of iPS-derived liver cells and constructed three-dimensional tissues. In one embodiment, metabolic ability is evaluated by the method for detecting enzyme activity of CYP enzyme group in cells, according to the above-mentioned embodiment.

In the field of drug discovery, there is a demand for non-destructive, time-course evaluation of drug efficacy and toxicity (ADMET evaluation) using cell tissues. In one embodiment, such evaluation of drug efficacy and toxicity is performed by the method for detecting the enzyme activity of the CYP enzyme group in cells, according to the above-mentioned embodiment.

In the field of diagnosis, there is a demand for effective indicators that can identify abnormal sites in cells or tissues to be diagnosed, as well as technique that can rapidly detect and measure the indicators. For example, tissue diagnosis during and after surgery in cancer resection requires advanced judgment by a pathologist. In the above-mentioned embodiment, biomolecules involved in metabolism including CYP activity are imaged non-destructively and without staining. In one embodiment, efficacy toxicity evaluation, quality evaluation, and rapid tissue diagnosis of cells or tissues are performed by this imaging.

The present invention is not limited to the above-mentioned embodiments, and can be modified as appropriate without departing from the gist.

### INDUSTRIAL APPLICABILITY

As described above, the present invention can be extremely useful in the fields of regenerative medical technology, drug discovery technology, and diagnostic technology.

This application is based on a patent application No. 2021-104309 filed in Japan (filing date: Jun. 23, 2021), the contents of which are incorporated in full herein.

1. A method for evaluating enzyme activity of CYP enzyme group inside or outside a cell, comprising a step of measuring the number of molecules of oxidized CYP enzyme group.

2. The method according to claim 1, wherein the method is for evaluating intracellular enzyme activity of CYP enzyme group, and wherein the step of measuring the number of molecules of the oxidized CYP enzyme group comprises
a step of irradiating an excitation light on the cells and obtaining a Raman spectrum using a photodetector, and
a step of extracting Raman scattering signals derived from the CYP enzyme group from the aforementioned Raman spectrum.

3. The method according to claim 2, wherein a wavenumber of the aforementioned Raman scattering signals derived from the CYP enzyme group is in the range of wavenumber 300-600, 620-880, 920-1320, or 1320-1660 cm⁻¹.

4. The method according to claim 3, wherein the aforementioned wavenumber is 1370 cm⁻¹ or 1636 cm⁻¹.

5. The method according to claim 1, wherein the aforementioned cell is derived from any of liver, small intestine, kidney, and brain.

6. The method according to claim 5, wherein the aforementioned cell is derived from the liver.

7. The method according to claim 1, wherein the aforementioned cell is derived from a pluripotent stem cell.

8. The method according to claim 1, further comprising a step of observing at least one selected from the group consisting of cell shape, cell size, and intracellular distribution of intracellular components in the region where the number of molecules of the CYP enzyme group is measured.

9. The method according to claim 1, wherein the method comprises a step of further extracting Raman scattering signals derived from a substance other than the CYP enzyme group.

10. The method according to claim 9, wherein the aforementioned substance other than the CYP enzyme group is at least one selected from the group consisting of reduced heme b, reduced/oxidized heme c, glycogen, reduced/oxidized cytochrome c, phenylalanine, and lipid.

11. A method for evaluating a metabolic ability of hepatocytes, comprising the following steps:
a step of irradiating an excitation light on hepatocytes and obtaining a Raman spectrum using a photodetector, and
a step of detecting Raman signals of a biomolecule relating to metabolic ability of hepatocyte from the aforementioned Raman spectrum.

12. The method according to claim 11, wherein the aforementioned biomolecule relating to metabolic ability of hepatocyte is at least one selected from the group consisting of CYP enzyme group, glycogen, cytochrome b₅, cytochrome c, lipid, and phenylalanine.
